(19) United States
(12) Patent Application Publication
(10) Pub. No.: US 2023/0381523 A1
(43) Pub. Date: Nov. 30, 2023

(54) SINGLE CHANNEL SENSING USING VFA DEVICE

(71) Applicant: Medtronic, Inc., Minneapolis, MN (US)

(72) Inventors: Yanina Grinberg, Plymouth, MN (US); Troy E. Jackson, Rogers, MN (US); Eric R. Williams, Maple Grove, MN (US); John C. Stroebel, Blaine, MN (US); Karen J. Kleckner, Bradenton, FL (US); Todd J. Sheldon, North Oaks, MN (US); Mackenzie E. Hall, Minneapolis, MN (US); Saul E. Greenhut, Denver, CO (US)

(21) Appl. No.: 17/824,764

(22) Filed: May 25, 2022

(51) Int. Cl. A61N 1/368; A61N 1/375; A61N 1/05; A61N 1/372

(52) U.S. Cl. CPC A61N 1/368; A61N 1/3756; A61N 1/37512; A61N 1/0573; A61N 1/37205; A61N 1/37518

(57) ABSTRACT

Ventricle-from-atrium (VfA) devices, systems, and methods may be configured to monitor a single channel of cardiac electrical activity and determine atrial events and ventricular events based on the single channel of cardiac electrical activity. Various processing, windowing, and thresholding may be used to identify atrial events and ventricular events within the single channel of cardiac electrical activity.

The disclosure herein relates to single channel sensing using ventricle-from-atrium (VfA) devices, systems, and methods.

The cardiac conduction system includes the sinus atrial (SA) node, the atrioventricular (AV) node, the bundle of His, bundle branches and Purkinje fibers. A heartbeat is initiated in the SA node, which may be described as the natural “pacemaker” of the heart. An electrical impulse arising from the SA node causes the atrial myocardium to contract. The signal is conducted to the ventricles via the AV node which inherently delays the conduction to allow the atria to stop contracting before the ventricles begin contracting thereby providing proper AV synchrony. The electrical impulse is conducted from the AV node to the ventricular myocardium via the bundle of His, bundle branches, and Purkinje fibers.

Patients with a conduction system abnormality, such as poor AV node conduction or poor SA node function, may receive an implantable medical device (IMD), such as a pacemaker, to restore a more normal heart rhythm and AV synchrony. Some types of IMDs, such as cardiac pacemakers, implantable cardioverter defibrillators (ICDs), or cardiac resynchronization therapy (CRT) devices, provide therapeutic electrical stimulation to a heart of a patient via electrodes on one or more implantable endocardial, epicardial, or coronary venous leads that are positioned in or adjacent to the heart. The therapeutic electrical stimulation may be delivered to the heart in the form of pulses or shocks for pacing, cardioversion, or defibrillation. In some cases, an IMD may sense intrinsic depolarizations of the heart, and control the delivery of therapeutic stimulation to the heart based on the sensing.

Delivery of therapeutic electrical stimulation to the heart can be useful in addressing cardiac conditions such as ventricular dyssynchrony that may occur in patients. Ventricular dyssynchrony may be described as a lack of synchrony or a difference in the timing of contractions in different ventricles of the heart. Significant differences in timing of contractions can reduce cardiac efficiency. CRT, delivered by an IMD to the heart, may enhance cardiac output by resynchronizing the electromechanical activity of the ventricles of the heart. CRT is sometimes referred to as “triple chamber pacing” because CRT provides pacing to the right atrium, right ventricle, and left ventricle.

Cardiac arrhythmias may be treated by delivering electrical shock therapy for cardioverting or defibrillating the heart, for example, using an IMD or an ICD, each of which may sense a patient's heart rhythm and classify the rhythm according to an arrhythmia detection scheme in order to detect episodes of tachycardia or fibrillation. Arrhythmias detected may include ventricular tachycardia (VT), fast ventricular tachycardia (FVT), ventricular fibrillation (VF), atrial tachycardia (AT) and atrial fibrillation (AF). Anti-tachycardia pacing (ATP), a painless therapy, can be used to treat ventricular tachycardia (VT) to substantially terminate many monomorphic fast rhythms. While ATP is painless, ATP may not deliver effective therapy for all types of VTs and for supraventricular tachycardia (SVT). For example, ATP may not be as effective for polymorphic VTs, which has variable morphologies. Polymorphic VTs and ventricular fibrillation (VFs) can be more lethal and may require expeditious treatment by shock.

Dual chamber medical devices are available that include a transvenous atrial lead carrying electrodes that may be placed in the right atrium and a transvenous ventricular lead carrying electrodes that may be placed in the right ventricle via the right atrium. The dual chamber medical device itself is generally implanted in a subcutaneous pocket and the transvenous leads are tunneled to the subcutaneous pocket. A dual chamber medical device may sense atrial electrical signals and ventricular electrical signals and can provide both atrial pacing and ventricular pacing as needed to promote a normal heart rhythm and AV synchrony. Some dual chamber medical devices can treat both atrial and ventricular arrhythmias.

Intracardiac medical devices, such as a leadless pacemaker, have been introduced or proposed for implantation entirely within a patient's heart, eliminating the need for transvenous leads. A leadless pacemaker may include one or more electrodes on its outer housing to deliver therapeutic electrical signals and/or sense intrinsic depolarizations of the heart. Intracardiac medical devices may provide cardiac therapy functionality, such as sensing and pacing, within a single chamber of the patient's heart. Single chamber intracardiac devices may also treat either atrial or ventricular arrhythmias or fibrillation. Some leadless pacemakers are not intracardiac and may be positioned outside of the heart and, in some examples, may be anchored to a wall of the heart via a fixation mechanism.

In some patients, single chamber devices may adequately address the patient's needs. However, single chamber devices capable of only single chamber sensing and therapy may not fully address cardiac conduction disease or abnormalities in all patients, for example, those with some forms of AV dyssynchrony or tachycardia. Dual chamber sensing and/or pacing functions, in addition to ICD functionality in some cases, may be used to restore more normal heart rhythms.

### SUMMARY

The illustrative ventricle-from-atrium (VfA) devices, systems, and methods may be described as providing single channel sensing of atrial and ventricular events (e.g., contractions, depolarizations, etc.). VfA devices can include a plurality of electrodes, and often include at least a right atrial electrode (e.g., in contact with myocardial tissue of the right atrium, within the right atrial blood pool, etc.) to, at least, sense electrical activity of the right atrium (e.g., from the myocardial tissue, from the blood pool, etc.) and a tissue-piercing ventricular electrode configured to be located in the myocardial tissue of the left ventricle to, at least, sense electrical activity of the myocardial tissue of the left ventricle. Furthermore, in one or more alternative configurations, the tissue-piercing electrode may be configured to be located in the myocardial tissue of the right ventricle to, at least, sense electrical activity of the myocardial tissue of the right ventricle. Additionally, VfA devices often include a right atrial blood pool electrode that is positioned, or configured to be positioned, in the blood pool of the right atrium. Two or more of the of the right atrial electrode, the tissue-piercing ventricular electrode, and the right atrial blood pool electrode may be utilized, or used, to form, or define, a single sensing channel to sense cardiac electrical activity. Depending on the electrodes used to form, or define, the single sensing channel, various processes as will be described further herein may be utilized to identify, discriminate, or determine atrial and ventricular events. Use of a single channel as opposed to multiple channels may increase efficiency and may reduce complexity of the illustrative VfA devices, systems, and methods.

One illustrative implantable medical device may include, among other things a plurality of electrodes, a therapy delivery circuit operably coupled to the one or more electrodes to deliver cardiac therapy to the patient's heart, a sensing circuit operably coupled to the one or more electrodes to sense electrical activity of the patient's heart, and a controller comprising processing circuitry operably coupled to the therapy delivery circuit and the sensing circuit. The plurality of electrodes may include a right atrial electrode positionable within the right atrium of a patient's heart (e.g., in contact with myocardial tissue of the right atrium, within the right atrial blood pool, etc.) and a tissue-piercing electrode implantable through the right atrium from the triangle of Koch region of the right atrium through the right atrial endocardium and central fibrous body and positionable in contact with one or more of the basal region, septal region, and basal-septal region of the left ventricular myocardium of the patient's heart. The controller may be configured to monitor a single channel of electrical activity using one or both of the right atrial electrode positionable within the right atrium to sense electrical activity in the right atrium and the tissue-piercing electrode implantable in one or more of the basal region, septal region, and basal-septal region of the left ventricular myocardium of the patient's heart from the triangle of Koch region of the right atrium through the right atrial endocardium and central fibrous body to sense electrical activity in one or more of the basal region, septal region, and basal-septal region of the left ventricular myocardium; and identify atrial and ventricular events based on the monitored electrical activity.

One illustrative method may include obtaining a single channel of electrical activity using one or both of a right atrial electrode positioned within the right atrium (e.g., in contact with myocardial tissue of the right atrium, within the right atrial blood pool, etc.) to sense electrical activity in the right atrium and a tissue-piercing electrode implanted in one or more of the basal region, septal region, and basal-septal region of the left ventricular myocardium of the patient's heart from the triangle of Koch region of the right atrium through the right atrial endocardium and central fibrous body to sense electrical activity in one or more of the basal region, septal region, and basal-septal region of the left ventricular myocardium and identifying atrial and ventricular events based on the obtained electrical activity.

One illustrative implantable medical device may include a housing wholly implantable in the right atrium of a patient's heart, a plurality of electrodes leadlessly fixed to the housing, and a controller comprising processing circuitry operably coupled the plurality of electrodes and within the housing. The plurality of electrodes may include a right atrial electrode positionable within the right atrium (e.g., in contact with myocardial tissue of the right atrium, within the right atrial blood pool, etc.) and a tissue-piercing electrode implantable through a patient's right atrium so as to extend toward the left ventricular myocardium of the patient's heart. The controller may be configured to monitor a single channel of electrical activity using one or both of the right atrial electrode positionable within the right atrium and the tissue-piercing electrode implantable through a patient's right atrium so as to extend toward the left ventricular myocardium of the patient's heart and identify atrial and ventricular events based on the monitored electrical activity.

One illustrative a method may include obtaining, with a fully intracardiac leadless device, a single channel of electrical activity using a right atrial electrode of the leadless device positioned within the right atrium of the patient's heart and a tissue-piercing electrode of the leadless device that extends toward the left ventricular myocardium of the patient's heart and identifying atrial and ventricular events based on the obtained electrical activity.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a conceptual diagram of an illustrative cardiac therapy system including a VfA device implanted in a patient's heart and a separate medical device positioned outside of the patient's heart.

FIG. 2 is an enlarged conceptual diagram of the VfA device of FIG. 1 positioned with respect to anatomical structures of the patient's heart.

FIG. 3 is a conceptual diagram of a map of a patient's heart in a standard 17 segment view of the left ventricle showing various electrode implantation locations for use with the illustrative systems, devices, and methods described herein.

FIG. 4 is a perspective view of the VfA device of FIG. 1 having a distal fixation and electrode assembly that includes a distal housing-based electrode implemented as a ring electrode.

FIG. 5 is a perspective view of another illustrative VfA device.

FIG. 6 is a block diagram of illustrative circuitry that may be enclosed within the housing of the VfA devices of FIGS. 1-2 and 4-5, for example, to provide the functionality and therapy thereof.

FIG. 7 is a block diagram of an illustrative method of single channel sensing, for example, performable by the illustrative systems and devices of FIGS. 1-2 and 4-6.

FIG. 8 is a block diagram of another illustrative method of single channel sensing, for example, performable by the illustrative systems and devices of FIGS. 1-2 and 4-6.

FIGS. 9A-9E are graphs of a single channel of electrical activity measured using a right atrial electrode and a right atrial blood pool electrode of an illustrative VfA device showing the method of FIG. 7.

FIGS. 10A-10B are graphs of a single channel of electrical activity measured using a tissue-piercing electrode and a right atrial blood pool electrode of an illustrative VfA device showing the method of FIG. 8.

FIGS. 11A-11B are graphs of a single channel of electrical activity measured using a tissue-piercing electrode and a right atrial electrode of an illustrative VfA device showing the method of FIG. 8.

FIG. 12 is a block diagram of another illustrative method of single channel sensing, for example, performable by the illustrative systems and devices of FIGS. 1-2 and 4-6.

### DETAILED DESCRIPTION OF ILLUSTRATIVE EMBODIMENTS

In the following detailed description of illustrative embodiments, reference is made to the accompanying figures of the drawing which form a part hereof, and in which are shown, by way of illustration, specific embodiments which may be practiced. It is to be understood that other embodiments may be utilized, and structural changes may be made without departing from (e.g., still falling within) the scope of the disclosure presented hereby.

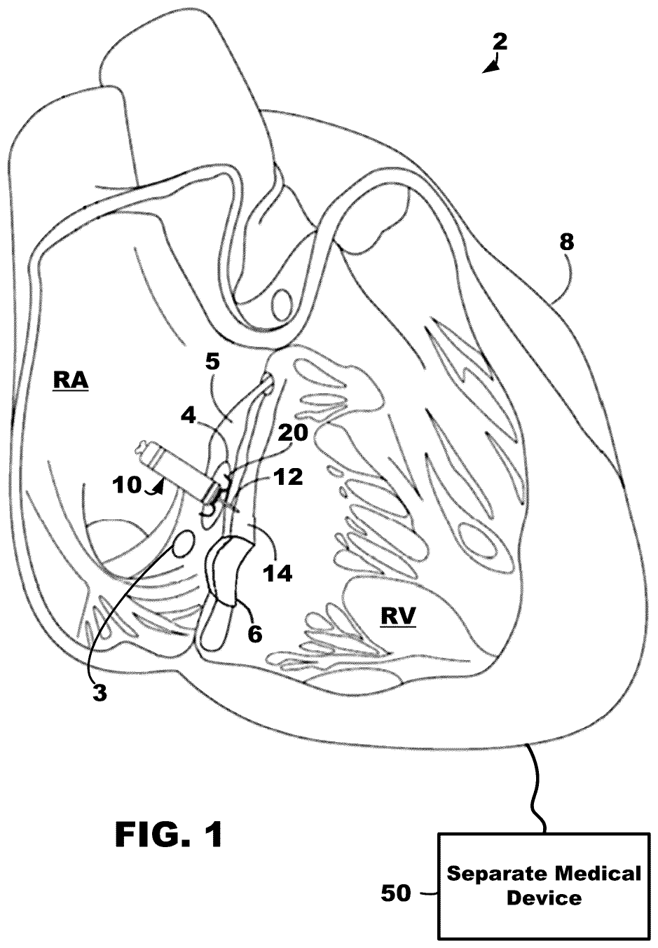

Illustrative devices, systems, and methods shall be described with reference to FIGS. 1-12. It will be apparent to one skilled in the art that elements or processes from one embodiment may be used in combination with elements or processes of the other embodiments, and that the possible embodiments of such devices, systems, and methods using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

The present disclosure describes implantable pacing devices, and in particular, VfA implantable pacing devices, that are configured to sense atrial and ventricular events utilizing a single sensing channel, which may be used to provide one or more various pacing therapies such as, for example, AV synchronous pacing therapy, cardiac resynchronization therapy, antitachycardia pacing therapy, defibrillation, cardioversion, etc. The implantable devices may be paired with another device such as, for example, an extravascular implantable cardioverter defibrillator (EV-ICD) to provide cardioversion and defibrillation.

As will be described further herein, some embodiments of the illustrative VfA implantable medical devices (IMDs) may be described as dual chamber leadless pacemakers implanted in the Triangle of Koch (TOK) region of the right atrium. In one embodiment, a VfA IMD may include three electrodes: a tissue-piercing ventricular electrode positioned in the left ventricular myocardium; a right atrial electrode contacting atrial myocardial tissue at the TOK; and a right atrial blood pool electrode. Generally, one or both of the tissue-piercing ventricular electrode and the right atrial electrodes may be utilized as a cathode and the right atrial blood pool electrode may be utilized as an anode for sensing and pacing.

Often, dual chamber pacing IMDs may utilize two independent sensing channels with a pair of sensing electrodes located in the right atrium for sensing atrial signals and another a pair of sensing electrodes located proximate (e.g., within) a ventricle for sensing ventricular signals. In a two independent sensing channel configuration, far-field signals, that is signals originating from a chamber outside of where a sensing electrode (and more particularly, the cathode of a sensing pair of electrodes) is placed, are minimized by a common mode rejection of the closely spaced bipole (e.g., the pair of sensing electrodes) and the relatively large distance of the atrial and ventricular electrodes, respectively, from the source of the far-field signals.

VfA IMDs may sense more or have larger far-field signals than traditional dual chamber pacing IMDs due to the proximity of the electrodes to tissue providing far-field signals such as, e.g., the right atrial tip electrode being in proximity to ventricular tissue, the right atrial blood pool electrode (e.g., a common anode ring in the right atrium) being in proximity to ventricular tissue, and the tissue-piercing left ventricular electrode being in proximity to atrial tissue. As a result, a sensed atrial signal using a VfA IMD may be contaminated with far-field R-waves and a sensed ventricular signal using a VfA IMD may be contaminated with far-field P-waves. Far-field R-waves may be undesirable when analyzing the sensed atrial signal for atrial events, and likewise, far-field P-waves may be undesirable when analyzing the sensed ventricular signal for ventricular events. Furthermore, it may be desirable for VfA IMDs to have, or define, maximal longevity (e.g., battery life) while also having a minimal battery or power source.

The illustrative VfA devices, systems, and methods described herein may utilize a single sensing channel to identify atrial and ventricular events to facilitate cardiac condition diagnostics and cardiac pacing therapy. The illustrative VfA devices, systems, and methods may be described as minimizing current drain associated with sensing, which may maximize the battery life and longevity of the VfA devices, minimizing hardware design complexity and space usage (e.g., if a single channel is utilized as opposed to two channels, one less sense amplifier and one less amplifier may be utilized), maximizing the duration of an electrogram episode that can be stored in constrained, or restricted, pool of memory, and minimizing current drain associated with telemetry when uplinking a single sensing channel as opposed to two sensing channels. Thus, illustrative devices, systems, and methods described herein may provide a single sensing channel that may be used to determine one or more cardiac conditions and to deliver, or configure, various cardiac therapy.

As described herein, this disclosure is related to implantable medical devices, systems, and methods for ventricle-from-atrium (VfA) cardiac therapy, including single chamber or multiple chamber pacing (e.g., dual or triple chamber pacing), atrioventricular synchronous pacing, asynchronous pacing, triggered pacing, cardiac resynchronization pacing, or tachycardia-related therapy. Although reference is made herein to implantable medical devices, and in particular, VfA IMDs, the methods and processes may be used with any medical devices and systems related to, or used to treat, a patient's heart. Various other applications will become apparent to one of skill in the art having the benefit of the present disclosure.

The present disclosure provides, among other things, an IMD including a tissue-piercing left ventricular electrode and a right atrial electrode. The tissue-piercing left ventricular electrode may be implanted in the basal, septal, and/or basal-septal region of the left ventricular myocardium of the patient's heart from the triangle of Koch region of the right atrium through the right atrial endocardium and central fibrous body. In a leadless IMD, the tissue-piercing electrode may leadlessly extend from a distal end region of a housing of the device, and the right atrial electrode may be leadlessly coupled to the housing (e.g., part of or positioned on the exterior of the housing). In a leaded IMD, one or more of the electrodes may be coupled to the housing using an implantable lead. When the device is implanted, the electrodes may be used to sense electrical activity in one or more atria and/or ventricles of a patient's heart. The electrodes may be used to deliver cardiac therapy, such as single chamber pacing for atrial fibrillation, atrioventricular synchronous pacing for bradycardia, asynchronous pacing, triggered pacing, cardiac resynchronization pacing for ventricular dyssynchrony, anti-tachycardia pacing, or shock therapy. When used in conjunction with an extravascular or subcutaneous ICD, the illustrative IMD may be in operative communication therewith to trigger, or initiate, an electrical shock provided by the IMD.

It is to be understood that the processes and methods described herein may be implemented by one or more various devices (e.g., implantable medical devices) and systems. Such devices and systems may include electronic circuits, power sources, sensors, electrodes, fluid delivery devices, etc. One illustrative cardiac therapy system 2 including an implantable medical device (IMD) 10 that may be used in carrying out the methods and processes described herein is depicted in FIG. 1. Although it is to be understood that the present disclosure may utilize one or both of leadless and leaded implantable medical devices, the illustrative cardiac therapy system 2 a leadless IMD 10 implanted in a patient's heart 8.

The IMD 10 may be used, at least, to treat heart conditions by delivering electrical stimulation to one or more regions or areas of the heart 8. For example, the IMD 10 may deliver pacing pulses to one or more chambers of the heart such as the right atria and left ventricle. Further, for example, the IMD 10 may deliver antitachycardia pacing pulses to one or more chambers of the heart such as the right atria and left ventricle. Still further, for example, the IMD 10 may deliver cardioversion or defibrillation shock pulses to one or more portions of the heart.

And still further, for example, the IMD 10 may deliver pacing pulses to one or more portion of the cardiac conduction system such as the left bundle branch. In some embodiments, the device 10 may be configured for single chamber pacing and may, for example, switch between single chamber and multiple chamber pacing (e.g., dual or triple chamber pacing).

The device 10 is shown implanted in the right atrium (RA) of the patient's heart 8 in a target implant region 4. The device 10 may include one or more fixation members 20 that anchor a distal end of the device 10 against the atrial endocardium in a target implant region 4 within the triangle of Koch region. The device 10 may include one or more fixation members 20 that anchor a distal end of the device against the atrial endocardium in a target implant region 4. The target implant region 4 may lie between the His bundle 5 (or bundle of His) and the coronary sinus 3 and may be adjacent the tricuspid valve 6. The device 10 may be described as a ventricle-from-atrium (VfA) device, which may sense electrical activity or provide therapy to one or both ventricles (e.g., right ventricle, left ventricle, or both ventricles) while being generally disposed in the right atrium. In particular, the device 10 may include a tissue-piercing electrode that may be implanted in the basal, septal, and/or basal-septal regions of the left ventricular myocardium of the patient's heart from the triangle of Koch region of the right atrium through the right-atrial endocardium and central fibrous body.

The device 10 may be described as a leadless implantable medical device. As used herein, “leadless” refers to a device being free of a lead extending out of the patient's heart 8. Further, although a leadless device may have a lead, the lead would not extend from outside of the patient's heart to inside of the patient's heart or would not extend from inside of the patient's heart to outside of the patient's heart. Some leadless devices may be introduced through a vein, but once implanted, the device is free of, or may not include, any transvenous lead and may be configured to provide cardiac therapy without using any transvenous lead. Further, a leadless device, in particular, does not use a lead to operably connect to one or more electrodes when a housing of the device is positioned in the atrium. Additionally, a leadless electrode may be coupled to the housing of the medical device without using a lead between the electrode and the housing.

The device 10 may be configured to monitor one or more physiological parameters of a patient (e.g., electrical activity of a patient's heart, chemical activity of a patient's heart, hemodynamic activity of a patient's heart, and motion and acceleration of one or more portions of the patient's heart). The monitored physiological parameters, in turn, may be used by the IMD to detect various cardiac conditions, e.g., ventricular tachycardia (VT), ventricular fibrillation (VF), supraventricular ventricular tachycardia (SVT), atrial fibrillation (AF), atrial tachycardia (AT), myocardial ischemia/infarction, etc., and to treat such cardiac conditions with therapy. Such therapy may include delivering antitachycardia pacing (ATP) therapy, defibrillation or cardioversion shock therapy (e.g., delivering high-energy shock pulses), cardiac resynchronization therapy, AV synchronous pacing therapy, bradycardia pacing, etc. In particular, the IMD 10 may monitor atrial and ventricular electrical activity to determine, or identify, atrial and ventricular events (e.g., contractions, depolarizations, etc.), determine one or more cardiac conditions (e.g., tachyarrhythmias), and deliver therapy to the patient such as AV synchronous pacing therapy or CRT.

The device 10 may also include a dart electrode assembly 12 defining, or having, a straight shaft extending from a distal end region of device 10. The dart electrode assembly 12 may be primarily utilized to provide ventricular pacing and sensing and may be placed, or at least configured to be placed, through the atrial myocardium and the central fibrous body and into the ventricular myocardium 14, or along the ventricular septum, without perforating entirely through the ventricular endocardial or epicardial surfaces. The dart electrode assembly 12 may carry, or include, an electrode at a distal end region of the shaft such that the electrode may be positioned within the ventricular myocardium for sensing ventricular signals and delivering ventricular pacing pulses (e.g., to depolarize the left ventricle and/or right ventricle to initiate a contraction of the left ventricle and/or right ventricle). In some examples, the electrode at the distal end region of the shaft is a cathode electrode provided for use in a bipolar electrode pair for pacing and sensing. While the implant region 4 as illustrated may enable one or more electrodes of the dart electrode assembly 12 to be positioned in the ventricular myocardium, it is recognized that a device having the aspects disclosed herein may be implanted at other locations for multiple chamber pacing (e.g., dual or triple chamber pacing), single chamber pacing with multiple chamber sensing, single chamber pacing and/or sensing, or other clinical therapy and applications as appropriate.

It is to be understood that although device 10 is described herein as including a single dart electrode assembly, the device 10 may include more than one dart electrode assembly placed, or configured to be placed, through the atrial myocardium and the central fibrous body, and into the ventricular myocardium 14, or along the ventricular septum, without perforating entirely through the ventricular endocardial or epicardial surfaces. Additionally, each dart electrode assembly may carry, or include, more than a single electrode at the distal end region, or along other regions (e.g., proximal or central regions), of the shaft. In other words, each dart electrode assembly may include one or more electrodes at the distal end region of the shaft that could be used, e.g., for bipolar sensing, bipolar pacing, or additional sensing for pacing capture.

The cardiac therapy system 2 may also include a separate medical device 50 (depicted diagrammatically in FIG. 1), which may be positioned outside the patient's heart 8 (e.g., subcutaneously) and may be operably coupled to the patient's heart 8 to deliver cardiac therapy thereto. In one example, separate medical device 50 may be an extravascular ICD. In some embodiments, an extravascular ICD may include a defibrillation lead including, or carrying, a defibrillation electrode. A therapy vector may exist between the defibrillation electrode on the defibrillation lead and a housing electrode of the ICD. Further, one or more electrodes of the ICD may also be used for sensing electrical signals related to the patient's heart 8. The ICD may be configured to deliver shock therapy including one or more defibrillation or cardioversion shocks. For example, if a tachyarrhythmia is sensed, the ICD may send a pulse via the electrical lead wires to shock the heart and restore its normal rhythm. In some examples, the ICD may deliver shock therapy without placing electrical lead wires within the heart or attaching electrical wires directly to the heart (subcutaneous ICDs). Examples of extravascular, subcutaneous ICDs that may be used with the system 2 described herein may be described in U.S. Pat. No. 9,278,229 issued on Mar. 8, 2016, which is incorporated herein by reference in its entirety.

In the case of shock therapy (e.g., defibrillation shocks provided by the defibrillation electrode of the defibrillation lead), the separate medical device 50 (e.g., extravascular ICD) may include a control circuit that uses a therapy delivery circuit to generate defibrillation shocks having any of a number of waveform properties, including leading-edge voltage, tilt, delivered energy, pulse phases, and the like. The therapy delivery circuit may, for instance, generate monophasic, biphasic, or multiphasic waveforms. Additionally, the therapy delivery circuit may generate defibrillation waveforms having different amounts of energy. For example, the therapy delivery circuit may generate defibrillation waveforms that deliver a total of between approximately 60-80 Joules (J) of energy for subcutaneous defibrillation.

The separate medical device 50 may further include a sensing circuit. The sensing circuit may be configured to obtain electrical signals sensed via one or more combinations of electrodes and to process the obtained signals. The components of the sensing circuit may include analog components, digital components, or a combination thereof. The sensing circuit may, for example, include one or more sense amplifiers, filters, rectifiers, threshold detectors, analog-to-digital converters (ADCs), or the like. The sensing circuit may convert the sensed signals to digital form and provide the digital signals to the control circuit for processing and/or analysis. For example, the sensing circuit may amplify signals from sensing electrodes and convert the amplified signals to multi-bit digital signals by an ADC, and then provide the digital signals to the control circuit. In one or more embodiments, the sensing circuit may also compare processed signals to a threshold to detect the existence of atrial or ventricular depolarizations (e.g., P- or R-waves) and indicate the existence of the atrial depolarization (e.g., P-waves) or ventricular depolarizations (e.g., R-waves) to the control circuit.

The device 10 and the separate medical device 50 may cooperate to provide cardiac therapy to the patient's heart 8. For example, the device 10 and the separate medical device 50 may be used to detect tachyarrhythmias, monitor tachyarrhythmias, and/or provide tachyarrhythmia-related therapy. For example, the device 10 may communicate with the separate medical device 50 wirelessly to trigger shock therapy using the separate medical device 50. As used herein, “wirelessly” refers to an operative coupling or connection without using a metal conductor between the device 10 and the separate medical device 50. In one example, wireless communication may use a distinctive, signaling, or triggering electrical pulse provided by the device 10 that conducts through the patient's tissue and is detectable by the separate medical device 50. In another example, wireless communication may use a communication interface (e.g., an antenna) of the device 10 to provide electromagnetic radiation that propagates through patient's tissue and is detectable, for example, using a communication interface (e.g., an antenna) of the separate medical device 50.

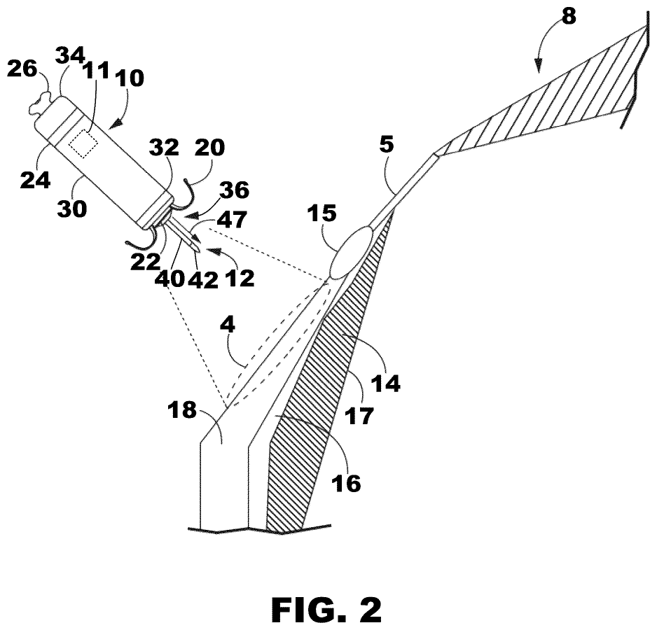

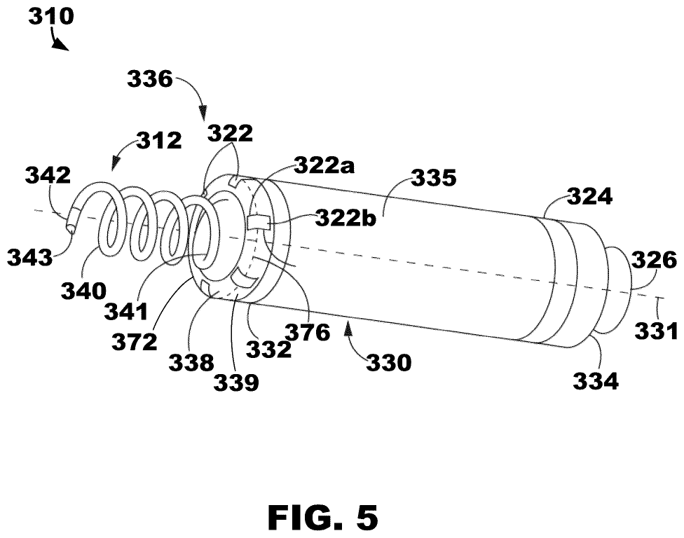

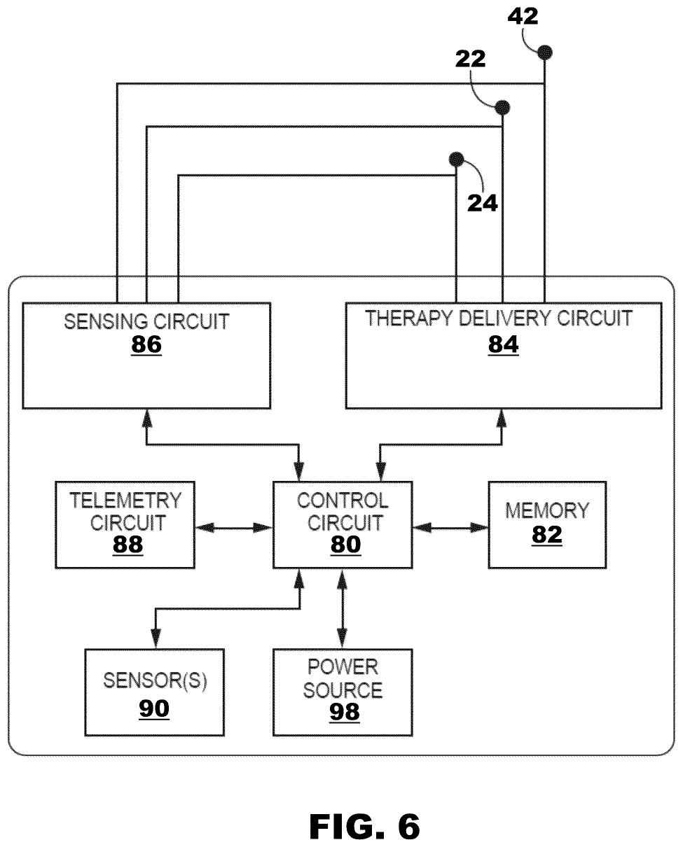

FIG. 2 is an enlarged conceptual diagram of the IMD 10 of FIG. 1 and anatomical structures of the patient's heart 8. As described herein, the IMD 10 is generally configured to sense cardiac signals and deliver pacing therapy. The IMD device 10 may include a housing 30 that defines a hermetically sealed internal cavity in which internal components of the device 10 reside, such as a sensing circuit, therapy delivery circuit, control circuit, memory, telemetry circuit, other optional sensors, and a power source as generally described in conjunction with FIG. 6. The housing 30 may include (e.g., be formed of or from) an electrically conductive material such as, e.g., titanium or titanium alloy, stainless steel, MP35N (a non-magnetic nickel-cobalt-chromium-molybdenum alloy), platinum alloy, or other bio-compatible metal or metal alloy. In other examples, the housing 30 may include (e.g., be formed of or from) a non-conductive material including ceramic, glass, sapphire, silicone, polyurethane, epoxy, acetyl co-polymer plastics, polyether ether ketone (PEEK), a liquid crystal polymer, or other biocompatible polymer.

In at least one embodiment, the housing 30 may be described as extending between a distal end region 32 and a proximal end region 34 and as defining a generally cylindrical shape, e.g., to facilitate catheter delivery. In other embodiments, the housing 30 may be prismatic or any other shape to perform the functionality and utility described herein. The housing 30 may include a delivery tool interface member 26, e.g., defined, or positioned, at the proximal end region 34, for engaging with a delivery tool during implantation of the device 10.

All or a portion of the housing 30 may function as a sensing and/or pacing electrode during cardiac therapy. In the example shown, the housing 30 includes a proximal housing-based electrode 24 that circumscribes a proximal portion (e.g., closer to the proximal end region 34 than the distal end region 32) of the housing 30. When the housing 30 is (e.g., defines, formed from, etc.) an electrically conductive material, such as a titanium alloy or other examples listed above, portions of the housing 30 may be electrically insulated by a non-conductive material, such as a coating of parylene, polyurethane, silicone, epoxy, or other biocompatible polymer, leaving one or more discrete areas of conductive material exposed to form, or define, the proximal housing-based electrode 24. When the housing 30 is (e.g., defines, formed from, etc.) a non-conductive material, such as a ceramic, glass or polymer material, an electrically conductive coating or layer, such as a titanium, platinum, stainless steel, or alloys thereof, may be applied to one or more discrete areas of the housing 30 to form, or define, the proximal housing-based electrode 24. In other examples, the proximal housing-based electrode 24 may be a component, such as a ring electrode, that is mounted or assembled onto the housing 30. The proximal housing-based electrode 24 may be electrically coupled to internal circuitry of the device 10, e.g., via the electrically conductive housing 30 or an electrical conductor when the housing 30 is a non-conductive material.

In the example shown, the proximal housing-based electrode 24 is located nearer to the housing proximal end region 34 than the housing distal end region 32, and therefore, may be referred to as a proximal housing-based electrode 24. In other examples, however, the proximal housing-based electrode 24 may be located at other positions along the housing 30, e.g., more distal relative to the position shown.

At the distal end region 32, the IMD 10 may include a distal fixation and electrode assembly 36, which may include one or more fixation members 20 and one or more dart electrode assemblies 12 of equal or unequal length. In one such example as shown, a single dart electrode assembly 12 includes a shaft 40 extending distally away from the housing distal end region 32 and one or more electrode elements, such as a tip electrode 42 at or near the free, distal end region of the shaft 40. The tip electrode 42 may have a conical or hemi-spherical distal tip with a relatively narrow tip diameter (e.g., less than about 1 millimeter (mm)) for penetrating into and through tissue layers without using a sharpened tip or needle-like tip having sharpened or beveled edges.

The dart electrode assembly 12 may be configured to pierce through one or more tissue layers to position the tip electrode 42 within a desired tissue layer such as, e.g., the ventricular myocardium. As such, the height, or length, 47 of the shaft 40 may correspond to the expected pacing site depth, and the shaft 40 may have a relatively high compressive strength along its longitudinal axis to resist bending in a lateral or radial direction when pressed against and into the implant region 4. If a second dart electrode assembly 12 is employed, its length may be unequal to the expected pacing site depth and may be configured to act as an indifferent electrode for delivering of pacing energy to and/or sensing signals from the tissue. In one embodiment, a longitudinal axial force may be applied against the tip electrode 42, e.g., by applying longitudinal pushing force to the proximal end 34 of the housing 30, to advance the dart electrode assembly 12 into the tissue within the target implant region. In at least one embodiment, the height 47, or length of the shaft 40 may be adjustable in relation to the housing 10 (e.g., which may be adjustable during implantation to deliver stimulation at the appropriate depth).

The shaft 40 may be described as longitudinally non-compressive and/or elastically deformable in lateral or radial directions when subjected to lateral or radial forces to allow temporary flexing, e.g., with tissue motion, but may return to its normally straight position when lateral forces diminish. Thus, the dart electrode assembly 12 including the shaft 40 may be described as being resilient. When the shaft 40 is not exposed to any external force, or to only a force along its longitudinal central axis, the shaft 40 may retain a straight, linear position as shown.

In other words, the shaft 40 of the dart electrode assembly 12 may be a normally straight member and may be rigid. In other embodiments, the shaft 40 may be described as being relatively stiff but still possessing limited flexibility in lateral directions. Further, the shaft 40 may be non-rigid to allow some lateral flexing with heart motion. However, in a relaxed state, when not subjected to any external forces, the shaft 40 may maintain a straight position as shown to hold the tip electrode 42 spaced apart from the housing distal end region 32 at least by a height, or length, 47 of the shaft 40.

The one or more fixation members 20 may be described as one or more “tines” having a normally curved position. The tines may be held in a distally extended position within a delivery tool. The distal tips of tines may penetrate the heart tissue to a limited depth before elastically, or resiliently, curving back proximally into the normally curved position (shown) upon release from the delivery tool. Further, the fixation members 20 may include one or more aspects described in, for example, U.S. Pat. No. 9,675,579, issued on Jun. 13, 2017, and U.S. Pat. No. 9,119,959 issued on Sep. 1, 2015, each of which is incorporated herein by reference in its entirety.

The distal fixation and electrode assembly 36 includes a distal housing-based electrode 22. In the case of using the device 10 as a pacemaker for multiple chamber pacing (e.g., dual or triple chamber pacing) and sensing, the tip electrode 42 may be used as a cathode electrode paired with the proximal housing-based electrode 24 or distal housing-based electrode 22 serving as a return anode electrode. In particular, the tip electrode 42 may be utilized in a single sensing channel, or vector, including one of the proximal housing-based electrode 24 and the distal housing-based electrode 22. As described herein the single sensing channel may be utilized to identify, or determine, atrial events (e.g., atrial depolarizations, atrial contractions, P-waves, etc.) and ventricular events (e.g., ventricular depolarizations, ventricular contractions, R-waves, etc.). The identified atrial and ventricular events may be used to control atrial pacing pulses (e.g., delivered in the absence of a sensed P-wave) using the distal housing-based electrode 22 as a cathode and the proximal housing-based electrode 24 as the return anode and to control atrial-synchronized ventricular pacing pulses delivered using the tissue-piercing tip electrode 42 as a cathode and the proximal housing-based electrode 24 or the distal housing-based electrode 22 as the return anode.

As shown in this illustration, the target implant region 4 in some pacing applications is along the atrial endocardium 18, generally inferior to the AV node 15 and the His bundle 5. The dart electrode assembly 12 may at least partially define the height, or length, 47 of the shaft 40 for penetrating through the atrial endocardium 18 in the target implant region 4, through the central fibrous body 16, and into the ventricular myocardium 14 without perforating through the ventricular endocardial surface 17. When the height, or length, 47 of the dart electrode assembly 12 is fully advanced into the target implant region 4, the tip electrode 42 may rest within the ventricular myocardium 14, and the distal housing-based electrode 22 may be positioned in intimate contact with or close proximity to the atrial endocardium 18. The dart electrode assembly 12 may have a total combined height, or length, 47, which includes the tip electrode 42 and the shaft 40) from about 3 mm to about 8 mm in various examples. The diameter of the shaft 40 may be less than about 2 mm, and may be about 1 mm or less, or even about 0.6 mm or less.

The IMD 10 may include an acoustic and/or motion detector 11 within the housing 30. The acoustic or motion detector 11 may be operably coupled to one or more of a control circuit 80, a sensing circuit 86, or a therapy delivery circuit 84 as described with respect to FIG. 6. The acoustic and/or motion detector 11 may be used to monitor mechanical activity, such as atrial mechanical activity (e.g., an atrial contraction) and/or ventricular mechanical activity (e.g., a ventricular contraction). In some embodiments, the acoustic and/or motion detector 11 may be used to detect right atrial mechanical activity. A non-limiting example of an acoustic and/or motion detector 11 includes one or both of an accelerometer and a microphone. In some embodiments, the mechanical activity detected by the acoustic and/or motion detector 11 may be used to supplement or replace electrical activity detected by one or more of the electrodes of the device 10. For example, the acoustic and/or motion detector 11 may be used in addition to, or as an alternative to, the proximal housing-based electrode 24.

The acoustic and/or motion detector 11 may also be used for rate response detection or to provide a rate-responsive IMD. Various techniques related to rate response may be described in U.S. Pat. No. 5,154,170 issued on Oct. 13, 1992, and U.S. Pat. No. 5,562,711 issued on Oct. 8, 1996, each of which is incorporated herein by reference in its entirety.

In various embodiments, acoustic and/or motion sensor 11 may be used as a heart sound (HS) sensor and may be implemented as a microphone and/or a 1-, 2- or 3-axis accelerometer. In one embodiment, the acoustic and/or motion sensor 11 is implemented as a piezoelectric crystal mounted within the housing 30 that is responsive to the mechanical motion associated with heart sounds. Examples of other embodiments of acoustical sensors that may be adapted for implementation with the techniques of the present disclosure may be described generally in U.S. Pat. Nos. 4,546,777, 6,869,404, 5,554,177, and 7,035,684, each of which is incorporated herein by reference in its entirety.

In other words, various types of acoustic and/or motion sensors 11 may be used. For example, the acoustic and/or motion sensor 11 may be described as being any implantable or external sensor responsive to one or more of the heart sounds, and thereby, capable of producing, or generating, an electrical analog signal correlated in time and amplitude to the heart sounds. The analog signal may then be processed, which may include digital conversion, by a HS sensing module to obtain HS parameters, such as amplitudes or relative time intervals, as derived by the HS sensing module or control circuit 80. The acoustic and/or motion sensor 11 and the HS sensing module may be incorporated in an IMD such as, e.g., device 10, capable of delivering CRT or another cardiac therapy being optimized or may be implemented in a separate device having wired or wireless communication with another IMD or an external programmer or computer used during a pace parameter optimization procedure as described herein.

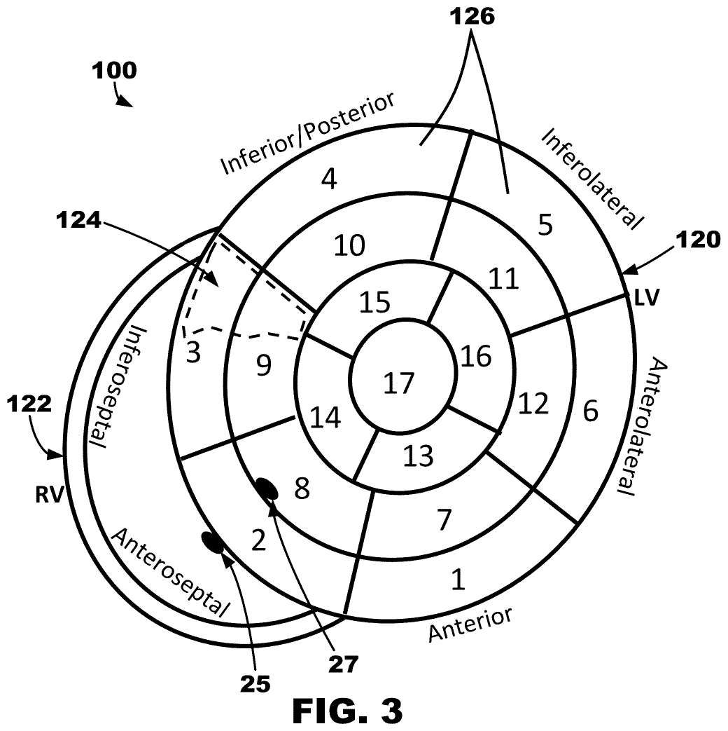

FIG. 3 is a two-dimensional (2D) ventricular map 100 of a patient's heart (e.g., a top-down view) showing the left ventricle 120 in a standard 17 segment view and the right ventricle 122. The map 100 defines, or includes, a plurality of areas 126 corresponding to different regions of a human heart. As illustrated, the areas 126 are numerically labeled 1-17 (which, e.g., correspond to a standard 17 segment model of a human heart, correspond to 17 segments of the left ventricle of a human heart, etc.). Areas 126 of the map 100 may include basal anterior area 1, basal anteroseptal area 2, basal inferoseptal area 3, basal inferior area 4, basal inferolateral area 5, basal anterolateral area 6, mid-anterior area 7, mid-anteroseptal area 8, mid-inferoseptal area 9, mid-inferior area 10, mid-inferolateral area 11, mid-anterolateral area 12, apical anterior area 13, apical septal area 14, apical inferior area 15, apical lateral area 16, and apex area 17. The inferoseptal and anteroseptal areas of the right ventricle 122 are also illustrated, as well as the right bunch branch (RBB) 25 and left bundle branch (LBB) 27.

In some embodiments, any of the tissue-piercing electrodes of the present disclosure may be implanted in the basal, septal, and/or basal-septal regions of the left ventricular myocardium of the patient's heart. In particular, the tissue-piercing electrode may be implanted from the triangle of Koch region of the right atrium through the right atrial endocardium and central fibrous body. Once implanted, the tissue-piercing electrode may be positioned in the target implant region 4 (FIGS. 1-2), such as the basal region, septal region, and/or basal-septal regions of the left ventricular myocardium. With reference to map 100, the basal region includes one or more of the basal anterior area 1, basal anteroseptal area 2, basal inferoseptal area 3, basal inferior area 4, mid-anterior area 7, mid-anteroseptal area 8, mid-inferoseptal area 9, and mid-inferior area 10. With reference to map 100, the septal region includes one or more of the basal anteroseptal area 2, basal anteroseptal area 3, mid-anteroseptal area 8, mid-inferoseptal area 9, and apical septal area 14. The basal-septal region may include one or more of the basal anteroseptal area 2, basal inferoseptal area 3, mid-anteroseptal area 8, and mid-inferoseptal area 9.

In some embodiments, the tissue-piercing electrode may be positioned in the high inferior/posterior basal septal region of the left ventricular myocardium when implanted. The high inferior/posterior basal septal region of the left ventricular myocardium may include a portion of one or more of the basal inferoseptal area 3 and mid-inferoseptal area 9 (e.g., the basal inferoseptal area only, the mid-inferoseptal area only, or both the basal inferoseptal area and the mid-inferoseptal area). For example, the high inferior/posterior basal septal region may include region 124 illustrated generally as a dashed-line boundary. As shown, the dashed line boundary represents an approximation of where the high inferior/posterior basal septal region is located, which may take a somewhat different shape or size depending on the particular application.

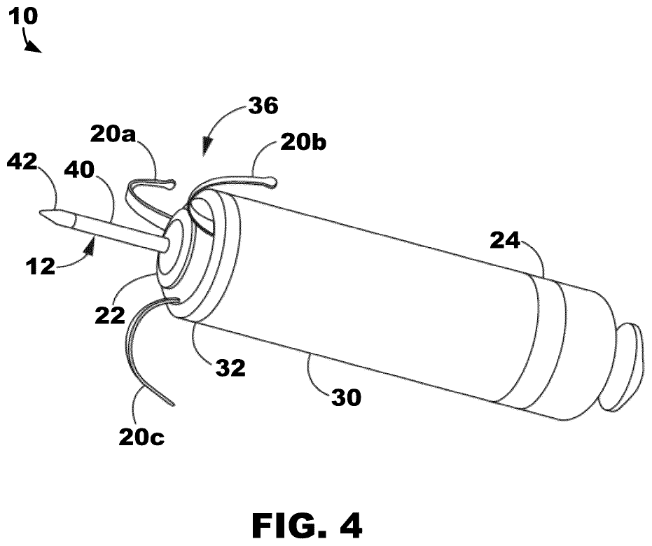

FIG. 4 is a three-dimensional perspective view of the device 10 capable of delivering pacing therapy and sensing cardiac signals. As shown, the distal fixation and electrode assembly 36 includes the distal housing-based electrode 22, implemented as a ring electrode. The distal housing-based electrode 22 may be positioned in intimate contact with or operative proximity to atrial tissue when fixation member tines 20a, 20b and 20c of the fixation members 20, engage with the atrial tissue. The tines 20a, 20b and 20c, which may be elastically deformable, may be extended distally during delivery of device 10 to the implant site. For example, the tines 20a, 20b, and 20c may pierce the atrial endocardial surface as the device 10 is advanced out of the delivery tool and flex back into their normally curved position (as shown) when no longer constrained within the delivery tool. As the tines 20a, 20b and 20c curve back into their normal position, the fixation member 20 may “pull” the distal fixation member and electrode assembly 36 toward the atrial endocardial surface. As the distal fixation member and electrode assembly 36 is “pulled” toward the atrial endocardium, the tip electrode 42 may be advanced through the atrial myocardium and the central fibrous body and into the ventricular myocardium. The distal housing-based electrode 22 may then be positioned against, or adjacent, the atrial endocardial surface.

The distal housing-based electrode 22 may include a ring formed of an electrically conductive material, such as titanium, platinum, iridium, or alloys thereof. The distal housing-based electrode 22 may be a single, continuous ring electrode. In other examples, portions of the ring may be coated with an electrically insulating coating, e.g., parylene, polyurethane, silicone, epoxy, or another insulating coating, to reduce the electrically conductive surface area of the ring electrode. For instance, one or more sectors of the ring may be coated to separate two or more electrically conductive exposed surface areas of the distal housing-based electrode 22. Reducing the electrically conductive surface area of the distal housing-based electrode 22, e.g., by covering portions of the electrically conductive ring with an insulating coating, may increase the electrical impedance of the distal housing-based electrode 22, and thereby, reduce the current delivered during a pacing pulse that captures the myocardium, e.g., the atrial myocardial tissue. A lower current drain may conserve the power source, e.g., one or more rechargeable or non-rechargeable batteries, of the device 10.

As described above, the distal housing-based electrode 22 may be configured as an atrial cathode electrode for delivering pacing pulses to the atrial tissue at the implant site in combination with the proximal housing-based electrode 24 as the return anode. Additionally, the distal housing-based electrode 22 may be utilized in a single sensing channel, or vector, including one of the proximal housing-based electrode 24 and the tissue-piercing electrode 42. As described herein the single sensing channel may be utilized to identify, or determine, atrial events (e.g., atrial depolarizations, atrial contractions, P-waves, etc.) and ventricular events (e.g., ventricular depolarizations, ventricular contractions, R-waves, etc.). The identified atrial and ventricular events may be used to control atrial pacing pulses (e.g., delivered in the absence of a sensed P-wave) using the distal housing-based electrode 22 as a cathode and the proximal housing-based electrode 24 as the return anode and to control atrial-synchronized ventricular pacing pulses delivered using the tissue-piercing tip electrode 42 as a cathode and the proximal housing-based electrode 24 or the distal housing-based electrode 22 as the return anode.

FIG. 5 is a three-dimensional perspective view of another leadless implantable medical device 310 that may be configured for treating heart conditions through delivering pacing therapy for single or multiple chamber cardiac therapy (e.g., dual or triple chamber cardiac therapy). The device 310 may include a housing 330 having an outer sidewall 335, shown as a cylindrical outer sidewall, extending from a housing distal end region 332 to a housing proximal end region 334. The housing 330 may enclose electronic circuitry configured to perform single or multiple chamber cardiac therapy, including atrial and ventricular cardiac electrical signal sensing and pacing the atrial and ventricular chambers. Delivery tool interface member 326 is shown on the housing proximal end region 334.

A distal fixation and electrode assembly 336 may be coupled to the housing distal end region 332. The distal fixation and electrode assembly 336 may include an electrically insulative distal member 372 coupled to the housing distal end region 332. The tissue-piercing electrode assembly 312 may extend away from the housing distal end region 332, and multiple non-tissue piercing electrodes 322 may be coupled directly to the insulative distal member 372. The tissue-piercing electrode assembly 312, as shown, extends in a longitudinal direction away from the housing distal end region 332 and may be coaxial with the longitudinal center axis 331 of the housing 330.

The distal tissue-piercing electrode assembly 312 may include an electrically insulated shaft 340 and a tip electrode 342 (e.g., tissue-piercing electrode). As described herein, embodiments may include a plurality of electrodes positioned along the insulated shaft 340. In some examples, the tissue-piercing electrode assembly 312 may be described as an active fixation member including a helical shaft 340 and a distal cathode tip electrode 342. The helical shaft 340 may extend from a shaft distal end region 343 to a shaft proximal end region 341, which may be directly coupled to the insulative distal member 372. The helical shaft 340 may be coated with an electrically insulating material, e.g., parylene or other examples listed herein, to avoid sensing or stimulation of cardiac tissue along the shaft length.

The tip electrode 342 is located, or positioned, at the shaft distal end region 343 and may serve as a cathode electrode for delivering ventricular pacing pulses and sensing ventricular electrical signals using the proximal housing-based electrode 324 or one or more of the non-tissue piercing electrodes 322 as a return anode when the tip electrode 342 is advanced proximate or into ventricular tissue as described herein. The proximal housing-based electrode 324 may be a ring electrode circumscribing the housing 330 and may be defined by an uninsulated portion of the longitudinal sidewall 335. Other portions of the housing 330 not serving as an electrode may be coated with an electrically insulating material similar to as described above in conjunction with the device 10 of FIG. 4.

Using two or more tissue-piercing electrodes (e.g., of any type) penetrating into the LV myocardium may be used for more localized pacing capture and may mitigate ventricular pacing spikes affecting capturing atrial tissue. In some embodiments, multiple tissue-piercing electrodes may include two or more dart-type electrode assemblies (e.g., electrode assembly 12 of FIG. 4), a helical-type electrode. Non-limiting examples of multiple tissue-piercing electrodes include two dart electrode assemblies, a helix electrode with a dart electrode assembly extending therethrough (e.g., through the center), or dual intertwined helixes. Multiple tissue-piercing electrodes may also be used for bipolar or multi-polar pacing.

In some embodiments, one or more tissue-piercing electrodes (e.g., of any type) that penetrate into the LV myocardium may be a multi-polar tissue-piercing electrode. A multi-polar tissue-piercing electrode may include one or more electrically active and electrically separate elements, which may enable bipolar or multi-polar pacing from one or more tissue-piercing electrodes. In other words, each tissue piercing electrode may include one or more separate electrodes or electrically active segments, or areas, that are independent from one another.

Multiple non-tissue piercing electrodes 322 may be provided along a periphery of the insulative distal member 372, peripheral to the tissue-piercing electrode assembly 312. The insulative distal member 372 may define a distal-facing surface 338 of the device 310 and a circumferential surface 339 that circumscribes the device 310 adjacent to the housing longitudinal sidewall 335. Non-tissue piercing electrodes 322 may be formed of an electrically conductive material, such as titanium, platinum, iridium, or alloys thereof. In the illustrated embodiment, six non-tissue piercing electrodes 322 are spaced apart radially at equal distances along the outer periphery of insulative distal member 372, however, two or more non-tissue piercing electrodes 322 may be provided.

Non-tissue piercing electrodes 322 may be discrete components each retained within a respective recess in the insulative member 372 sized and shaped to mate with the non-tissue piercing electrode 322. In other examples, the non-tissue piercing electrodes 322 may each be an uninsulated, exposed portion of a unitary member mounted within or on the insulative distal member 372. Intervening portions of the unitary member not functioning as an electrode may be insulated by the insulative distal member 372 or, if exposed to the surrounding environment, may be coated with an electrically insulating coating, e.g., parylene, polyurethane, silicone, epoxy, or other insulating coating.

When the tissue-piercing electrode assembly 312 is advanced into cardiac tissue, at least one non-tissue piercing electrode 322 may be positioned against, in intimate contact with, or in operative proximity to, a cardiac tissue surface for delivering pulses and/or sensing cardiac electrical signals produced by the patient's heart. For example, non-tissue piercing electrodes 322 may be positioned in contact with right-atrial endocardial tissue for pacing and sensing in the atrium when the tissue-piercing electrode assembly 312 is advanced into the atrial tissue and through the central fibrous body until the distal tip electrode 342 is positioned in direct contact with ventricular tissue, e.g., ventricular myocardium and/or a portion of the ventricular cardiac conduction system.

Non-tissue piercing electrodes 322 may be coupled to therapy delivery circuit and sensing circuit as will be described herein with respect to FIG. 6 enclosed by the housing 330. When delivering traditional pacing therapy, the non-tissue piercing electrodes 322 may operate to function collectively as a cathode electrode for delivering atrial pacing pulses and for sensing a single channel of electrical activity when used in combination with one of the proximal housing-based electrode 324 and the tip electrode 342 as a return anode. Switching circuitry included in a sensing circuit may be activated under the control of a control circuit to couple one or more of the non-tissue piercing electrodes to the single sensing channel. Distal, non-tissue piercing electrodes 322 may be electrically isolated from each other so that each individual one of the electrodes 322 may be individually selected by switching circuitry included in a therapy delivery circuit to serve alone or in a combination of two or more of the electrodes 322 as an atrial cathode electrode. Switching circuitry included in a therapy delivery circuit may be activated under the control of a control circuit to couple one or more of the non-tissue piercing electrodes 322 to an atrial pacing circuit. Two or more of the non-tissue piercing electrodes may be selected at a time to operate as a multi-point atrial cathode electrode.

Certain non-tissue piercing electrodes 322 selected for atrial pacing and/or atrial sensing may be selected based on atrial capture threshold tests, electrode impedance, P-wave signal strength in the cardiac electrical signal, or other factors. For example, a single one or any combination of two or more individual non-tissue piercing electrodes 322 functioning as a cathode electrode that provides an optimal combination of a low pacing capture threshold amplitude and relatively high electrode impedance may be selected to achieve reliable atrial pacing using minimal current drain from a power source.

In some instances, the distal-facing surface 338 may uniformly contact the atrial endocardial surface when the tissue-piercing electrode assembly 312 anchors the housing 330 at the implant site 4. In that case, all the electrodes 322 may be selected together to form the atrial cathode or anode. Alternatively, every other one of the electrodes 322 may be selected together to form a multi-point atrial cathode having a higher electrical impedance that is still uniformly distributed along the distal-facing surface 338. Alternatively, a subset of one or more electrodes 322 along one side of the insulative distal member 372 may be selected to provide pacing at a desired site that achieves the lowest pacing capture threshold due to the relative location of the electrodes 322 to the atrial tissue being paced.

In other instances, the distal-facing surface 338 may be oriented at an angle relative to the adjacent endocardial surface depending on the positioning and orientation at which the tissue-piercing electrode assembly 312 enters the cardiac tissue. In this situation, one or more of the non-tissue piercing electrodes 322 may be positioned in closer contact with the adjacent endocardial tissue than other non-tissue piercing electrodes 322, which may be angled away from the endocardial surface. By providing multiple non-tissue piercing electrodes along the periphery of the insulative distal member 372, the angle of the tissue-piercing electrode assembly 312 and the housing distal end region 332 relative to the cardiac surface, e.g., the right atrial endocardial surface, may not be required to be substantially parallel. Anatomical and positional differences may cause the distal-facing surface 338 to be angled or oblique to the endocardial surface, however, multiple non-tissue piercing electrodes 322 distributed along the periphery of the insulative distal member 372 increase the likelihood of good contact between one or more electrodes 322 and the adjacent cardiac tissue to promote acceptable pacing thresholds and reliable cardiac event sensing using at least a subset of multiple electrodes 322. Contact or fixation circumferentially along the entire periphery of the insulative distal member 372 may not be required.

The non-tissue piercing electrodes 322 may be described as including a first portion 322a extending along the distal-facing surface 338 and a second portion 322b extending along the circumferential surface 339. The first portion 322a and the second portion 322b may be continuous exposed surfaces such that the active electrode surface wraps around a peripheral edge 376 of the insulative distal member 372 that joins the distal facing surface 338 and the circumferential surface 339. The non-tissue piercing electrodes 322 may include one or more of the electrodes 322 along the distal-facing surface 338, one or more electrodes along the circumferential surface 339, one or more electrodes each extending along both of the distal-facing surface 338 and the circumferential surface 339, or any combination thereof. The exposed surface of each of the non-tissue piercing electrodes 322 may be flush with respective distal-facing surfaces 338 and/or circumferential surfaces. In other examples, each of the non-tissue piercing electrodes 322 may have a raised surface that protrudes from the insulative distal member 372. Any raised surface of the electrodes 322, however, may define a smooth or rounded, non-tissue piercing surface.

The distal fixation and electrode assembly 336 may seal the distal end region of the housing 330 and may provide a foundation on which the electrodes 322 are mounted. The electrodes 322 may be referred to as housing-based electrodes. The electrodes 322 may not be carried by a shaft or other extension that extends the active electrode portion away from the housing 330, like the distal tip electrode 342 residing at the distal tip of the helical shaft 340 extending away from the housing 330. Other examples of non-tissue piercing electrodes presented herein that are coupled to a distal-facing surface and/or a circumferential surface of an insulative distal member include the distal housing-based electrode 22 as described herein with respect to device 10 of FIG. 4, the distal housing-based electrode extending circumferentially around the assembly 36 as described herein with respect to device 10 of FIG. 4, button electrodes, other housing-based electrodes, and other circumferential ring electrodes. Any non-tissue piercing electrodes directly coupled to a distal insulative member, peripherally to a central tissue-piercing electrode, may be provided to function individually, collectively, or in any combination as a cathode electrode for delivering pacing pulses to adjacent cardiac tissue. When a ring electrode, such as the distal housing-based electrode 22 and/or a circumferential ring electrode, is provided, portions of the ring electrode may be electrically insulated by a coating to provide multiple distributed non-tissue piercing electrodes along the distal-facing surface and/or the circumferential surface of the insulative distal member.

The non-tissue piercing electrodes 322 and other examples listed above are expected to provide more reliable and effective atrial pacing and sensing than a tissue-piercing electrode provided along the distal fixation and electrode assembly 336. The atrial chamber walls are relatively thin compared to ventricular chamber walls. A tissue-piercing atrial cathode electrode may extend too deep within the atrial tissue leading to inadvertent sustained or intermittent capture of ventricular tissue. A tissue-piercing atrial cathode electrode may lead to interference with sensing atrial signals due to ventricular signals having a larger signal strength in the cardiac electrical signal received via tissue-piercing atrial cathode electrodes that are in closer physical proximity to the ventricular tissue. The tissue-piercing electrode assembly 312 may be securely anchored into ventricular tissue for stabilizing the implant position of the device 310 and providing reasonable certainty that the tip electrode 342 is sensing and pacing in ventricular tissue while the non-tissue piercing electrodes 322 may provide electrical stimulation to atrial tissue and sensing/monitoring of electrical activity of atrial tissue. The tissue-piercing electrode assembly 312 may be in the range of about 4 to about 8 mm in length from the distal-facing surface 338 to reach left ventricular tissue. In some instances, the device 310 may achieve four-chamber pacing by delivering atrial pacing pulses from the therapy delivery circuit 84 via the non-tissue piercing electrodes 322 in the target implant region 4 to achieve bi-atrial (right and left atrial) capture and by delivering ventricular pacing pulses from a ventricular pacing circuit via the tip electrode 342 advanced into ventricular tissue from the target implant region 4 to achieve biventricular (right and left ventricular) capture.

FIG. 6 is a block diagram of circuitry that may be enclosed within the housings 30, 330 of the devices 10, 310 to provide the functions of sensing cardiac signals, determining tachyarrhythmias, detecting tachyarrhythmias, discriminating tachyarrhythmias, determining supraventricular tachycardias, detecting supraventricular tachycardias, discriminating supraventricular tachycardias, determining capture of atrial and ventricular myocardial tissue, determining capture of one or more portions of the cardiac conduction system, and/or delivering pacing therapy to one or both of myocardial tissue and the cardiac conduction system. The separate medical device 50 as shown in FIG. 1 may include some or all the same components, which may be configured in a similar manner. The electronic circuitry enclosed within the housings 30, 330 may include software, firmware, and hardware that cooperatively monitor atrial and ventricular electrical cardiac signals, determine tachyarrhythmias, detect tachyarrhythmias, discriminate tachyarrhythmias, determine supraventricular tachycardias, detect supraventricular tachycardias, discriminate supraventricular tachycardias, determine whether cardiac conduction system capture has occurred, determine when a cardiac therapy such as ATP, defibrillation, and cardioversion may be delivered or inhibited, and deliver electrical pulses to the patient's heart according to programmed therapy mode and pulse control parameters. The electronic circuitry may include a control circuit 80 (e.g., including processing circuitry), a memory 82, a therapy delivery circuit 84, a sensing circuit 86, and/or a telemetry circuit 88. In some examples, the devices 10, 310 includes one or more sensors 90 for producing signals that are correlated to one or more physiological functions, states, or conditions of the patient. For example, the sensor(s) 90 may include a patient activity sensor, for use in determining a need for pacing therapy and/or controlling a pacing rate. Further, for example, the sensor(s) 90 may include an inertial measurement unit (e.g., accelerometer) to measure motion. Further, for example, the sensor(s) 90 may include an acoustic sensor to monitor cardiac sounds. Still further, for example, the sensor(s) 90 may include a patient activity sensor, which may include an accelerometer. An increase in the metabolic demand of the patient due to increased activity as indicated by the patient activity sensor may be determined using the patient activity sensor. In other words, the devices 10, 310 may include other sensors 90 for sensing signals from the patient for use in determining whether to deliver and/or controlling electrical stimulation therapies delivered by the therapy delivery circuit 84.

The power source 98 may provide power to the circuitry of the devices 10, 310 including each of the components 80, 82, 84, 86, 88, 90 as needed. The power source 98 may include one or more energy storage devices, such as one or more rechargeable or non-rechargeable batteries. The connections (not shown) between the power source 98 and each of the components 80, 82, 84, 86, 88, 90 may be understood from the general block diagram illustrated to one of ordinary skill in the art. For example, the power source 98 may be coupled to one or more charging circuits included in the therapy delivery circuit 84 for providing the power used to charge holding capacitors included in the therapy delivery circuit 84 that are discharged at appropriate times under the control of the control circuit 80 for delivering pacing pulses and/or shock pulses. The power source 98 may also be coupled to components of the sensing circuit 86, such as sense amplifiers, analog-to-digital converters, switching circuitry, etc., sensors 90, the telemetry circuit 88, and the memory 82 to provide power to the various circuits.

The functional blocks shown represent functionality included in the devices 10, 310 and may include any discrete and/or integrated electronic circuit components that implement analog, and/or digital circuits capable of producing the functions attributed to the medical devices 10, 310 described herein. The various components may include processing circuitry, such as an application specific integrated circuit (ASIC), an electronic circuit, a processor (shared, dedicated, or group), and memory that execute one or more software or firmware programs, a combinational logic circuit, state machine, or other suitable components or combinations of components that provide the described functionality. The particular form of software, hardware, and/or firmware employed to implement the functionality disclosed herein will be determined primarily by the particular system architecture employed in the medical device and by the particular detection and therapy delivery methodologies employed by the medical device.

The memory 82 may include any volatile, non-volatile, magnetic, or electrical non-transitory computer readable storage media, such as random-access memory (RAM), read-only memory (ROM), non-volatile RAM (NVRAM), electrically erasable programmable ROM (EEPROM), flash memory, or any other memory device. Furthermore, the memory 82 may include a non-transitory computer readable media storing instructions that, when executed by one or more processing circuits, cause the control circuit 80 and/or other processing circuitry to monitor atrial and ventricular electrical activity, detect tachyarrhythmias, determine tachyarrhythmias, discriminate tachyarrhythmias, detect supraventricular tachycardias, determine supraventricular tachycardias, discriminate supraventricular tachycardias, determine whether atrial electrical activity is reliable, determinate ventricular events (e.g., ventricular contractions), determinate atrial events (e.g., atrial contractions), compare QRS morphologies to templates indicative of normal sinus rhythm (e.g., templates of QRS morphologies of cardiac electrical activities during normal sinus rhythm), analyze one or more motion signals to determine whether the acceleration of the atria and ventricles, and/or perform a single, dual, or triple chamber calibrated pacing therapy (e.g., single or multiple chamber pacing), or other cardiac therapy functions (e.g., sensing or delivering therapy), attributed to the devices 10, 310. The non-transitory computer-readable media storing the instructions may include any of the media listed above.

The control circuit 80 may communicate, e.g., via a data bus, with the therapy delivery circuit 84 and the sensing circuit 86 for sensing cardiac electrical signals and controlling delivery of cardiac electrical stimulation therapies in response to the sensed cardiac activity (e.g., sensed atrial and ventricular events such as P-waves/atrial depolarizations and R-waves/ventricular depolarizations, or the absence thereof). The tip electrodes 42, 342, the distal housing-based electrodes 22, 322, and the proximal housing-based electrodes 24, 324 may be electrically coupled to the therapy delivery circuit 84 for delivering electrical stimulation pulses and to the sensing circuit 86 and for sensing electrical signals.

The distal housing-based electrodes 22, 322 and the proximal housing-based electrodes 24, 324 may be coupled to the sensing circuit 86 for sensing atrial signals, e.g., P-waves attendant to the depolarization of the atrial myocardium. In examples that include two or more selectable distal housing-based electrodes, the sensing circuit 86 may include switching circuitry for selectively coupling one or more of the available distal housing-based electrodes to event detection circuitry. Switching circuitry may include a switch array, switch matrix, multiplexer, or any other type of switching device suitable to selectively couple components of the sensing circuit 86 to selected electrodes. The tip electrodes 42, 324 and the proximal housing-based electrodes 24, 324 may be coupled to the sensing circuit 86 for sensing ventricular signals, e.g., R-waves attendant to the depolarization of the ventricular myocardium.

As described herein, the sensing circuit 86 may include event detection circuitry for detecting cardiac depolarization activity (e.g., P-waves, QRS complexes, R-waves, etc.). The event detection circuitry may be configured to amplify, filter, digitize, and rectify the electrical signals received from the selected electrodes to improve the signal quality for cardiac electrical events. The event detection circuitry may include one or more sense amplifiers, filters, rectifiers, threshold detectors, comparators, analog-to-digital converters (ADCs), timers, or other analog or digital components. Event sensing thresholds such as, e.g., P-wave sensing thresholds, R-wave sensing thresholds, etc. may be automatically adjusted under the control of the control circuit 80, e.g., based on timing intervals and sensing threshold values determined by the control circuit 80, stored in the memory 82, and/or controlled by hardware, firmware, and/or software of the control circuit 80 and/or the sensing circuit 86.

Upon detecting a cardiac electrical event based on a sensing threshold crossing, the sensing circuit 86 may produce a sensed event signal that is passed to the control circuit 80. For example, the sensing circuit 86 may produce a P-wave sensed event signal in response to a P-wave sensing threshold crossing and an R-wave sensed event signal in response to an R-wave sensing threshold crossing. The sensed event signals may be used by the control circuit 80 for setting pacing escape interval timers that control the basic time intervals used for scheduling cardiac pacing pulses. A sensed event signal may trigger or inhibit pacing pulses depending on the particular programmed pacing mode. For example, a P-wave sensed event signal received from the sensing circuit 86 may cause the control circuit 80 to inhibit a scheduled atrial pacing pulse and schedule a ventricular pacing pulse at a programmed atrioventricular (A-V) pacing interval. If an R-wave is sensed before the A-V pacing interval expires, the ventricular pacing pulse may be inhibited. If the A-V pacing interval expires before the control circuit 80 receives an R-wave sensed event signal from the sensing circuit 86, the control circuit 80 may use the therapy delivery circuit 84 to deliver the scheduled ventricular pacing pulse synchronized to the sensed P-wave.

In some examples, the devices 10, 310 may be configured to deliver a variety of therapies including bradycardia pacing, cardiac resynchronization therapy, post-shock pacing, and/or tachycardia-related therapy, such as ATP, among others. For example, the devices 10, 310 may be configured to detect non-sinus tachycardia and deliver antitachycardia pacing (ATP). The control circuit 80 may determine cardiac event time intervals, e.g., P-P intervals between consecutive P-wave sensed event signals received from the sensing circuit 86, R-R intervals between consecutive R-wave sensed event signals received from the sensing circuit 86, and P-R and/or R-P intervals received between P-wave sensed event signals and R-wave sensed event signals.

The therapy delivery circuit 84 may include charging circuitry, one or more charge storage devices such as one or more low voltage holding capacitors, an output capacitor, and/or switching circuitry that controls when the holding capacitor(s) are charged and discharged across the output capacitor to deliver electrical stimulation (e.g., cardiac pacing, defibrillation, cardioversion, etc.) to the one or more selected electrodes. The tip electrodes 42, 342, the proximal housing-based electrodes 24, 324, and the distal housing-based electrodes 22, 322 may be selectively coupled to the therapy delivery circuit 84 for delivery of atrial pacing pulses, ventricular pacing pulses, defibrillation and cardioversion shocks, etc. The therapy delivery circuit 84 may be configured to deliver ventricular pacing pulses, e.g., upon expiration of an A-V or V-V pacing interval set by the control circuit 80 for providing atrial-synchronized ventricular pacing and a basic lower ventricular pacing rate. The therapy delivery circuit 84 may be configured to deliver an atrial pacing pulse if the atrial pacing interval expires before a P-wave sensed event signal is received from the sensing circuit 86. The control circuit 80 starts an A-V pacing interval in response to a delivered atrial pacing pulse to provide synchronized multiple chamber pacing (e.g., dual or triple chamber pacing).

Charging of a holding capacitor of the therapy circuit 84 to a programmed pacing voltage amplitude and discharging of the capacitor for a programmed pacing pulse width may be performed according to control signals received from the control circuit 80. For example, a timing circuit included in the control circuit 80 may include programmable digital counters set by a microprocessor of the control circuit 80 for controlling the basic time intervals associated with various single chamber or multiple chamber pacing (e.g., dual or triple chamber pacing) modes and antitachycardia pacing sequences. The microprocessor of the control circuit 80 may also set the amplitude, pulse width, polarity, or other characteristics of the cardiac pacing pulses, which may be based on programmed values stored in the memory 82.

Control parameters utilized by the control circuit 80 for sensing cardiac events and controlling pacing therapy delivery may be programmed into the memory 82 via the telemetry circuit 88, which may also be described as a communication interface. The telemetry circuit 88 includes a transceiver and antenna for communicating with an external device, such as a programmer or home monitor, using radio frequency communication or other communication protocols. The control circuit 80 may use the telemetry circuit 88 to receive downlink telemetry from and send uplink telemetry to the external device. In some cases, the telemetry circuit 88 may be used to transmit and receive communication signals to/from another medical device implanted in the patient.

The illustrative devices, systems, and methods described herein may be used, or configured, to provide a single channel of cardiac electrical activity using at least one of a right atrial electrode such as, for example, the distal-housing based electrode 22, the non-tissue piercing electrode(s) 322, etc. and a tissue-piercing left ventricular electrode such as, e.g., tip electrode 42, tip electrode 342, etc. One or both of the right atrial electrode and the tissue-piercing left ventricular electrode may be utilized as the cathode in a sensing pair to provide the single channel of cardiac electrical activity. The anode may be a right atrial blood pool electrode such as, e.g., the proximal housing-based electrode 24, the proximal housing-based electrode 324, etc. Additionally, when the tissue-piercing left ventricular electrode is utilized as the cathode, the right atrial electrode may be used as the anode to form the sensing channel or vector. Further, it is to be understood that multiple point vectors (e.g., using more electrodes than a pair of electrodes) may be used to form, or define, the single sensing channel. In other words, more than two electrodes may be used to form, or define, the single sensing channel of cardiac electrical activity.

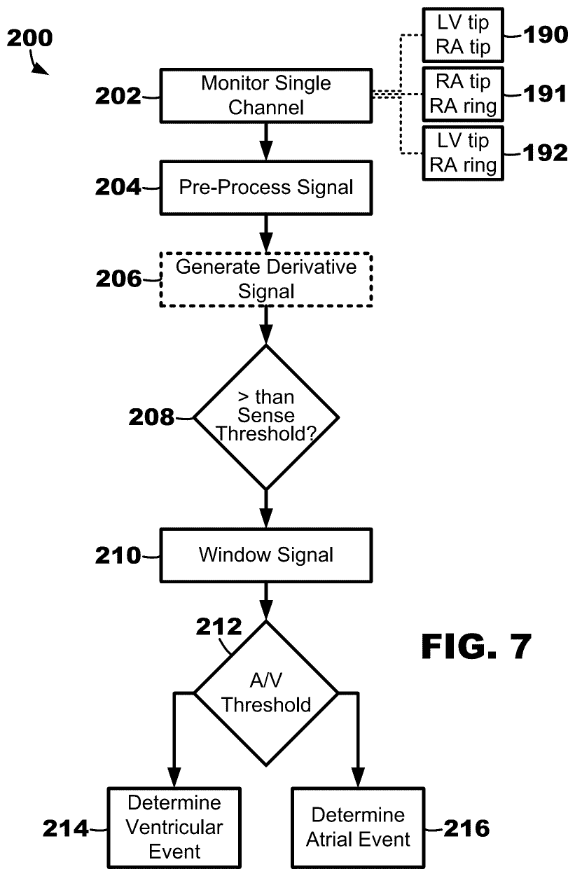

An illustrative method 200 of atrial and ventricular event identification utilizing a single channel of cardiac electrical activity is depicted in FIG. 7. Generally, it may be described that the illustrative method 200 collects, or obtains, a single sensing channel of cardiac electrical activity and processes the cardiac electrical activity to determine atrial and ventricular events therein.

As shown, the method 200 includes obtaining, or monitoring, the single channel of cardiac electrical activity using an electrode pair, or vector, such as a left ventricular electrode to distal right atrial electrode pair 190, a distal right atrial electrode to proximal right atrial electrode pair 191, and a left ventricular electrode to proximal right atrial electrode pair 192. It is to be understood that the left ventricular electrode can be any electrode configured to be positioned proximate (e.g., within) left ventricular myocardial tissue such as any of the tip electrode 42, tip electrode 342, and any other electrodes positioned on the dart electrode assembly 12 as described herein. Further, it is to be understood that the distal right atrial electrode can be any electrode configured to be positioned proximate (e.g., adjacent, in contact with, etc.) right atrial myocardial tissue such as any of the distal housing-based electrode 22 and the non-tissue piercing electrode(s) 322 as described herein. Still further, it is to be understood that the proximal right atrial electrode can be any electrode configured to be positioned proximate (e.g., within) the right atrial blood pool such as any of the proximal housing-based electrodes 24, 324 as described herein. Additionally, it is to be understood that more than a single pair of electrodes, such as a multipoint sensing vector, may be utilized to provide the single channel of cardiac electrical activity.

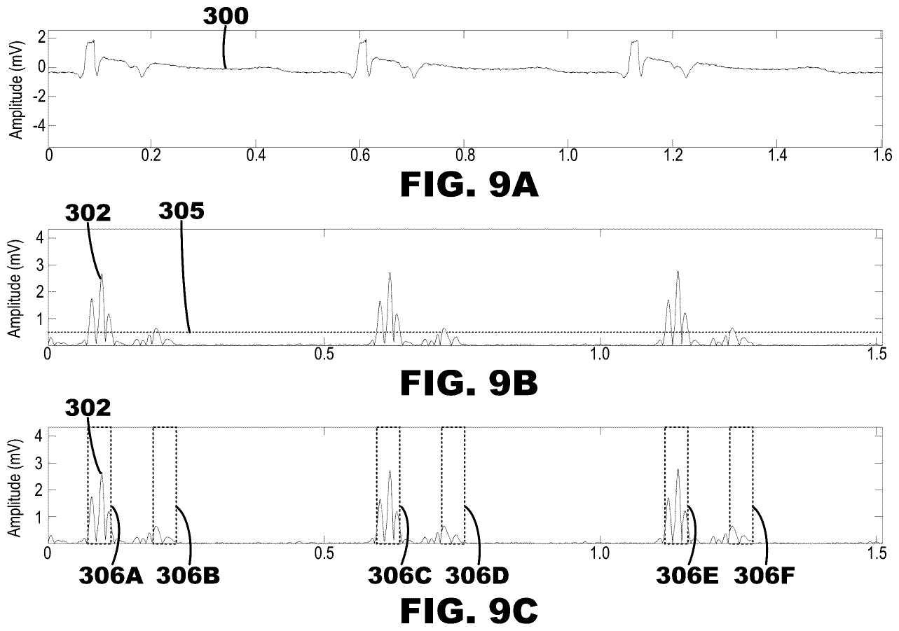

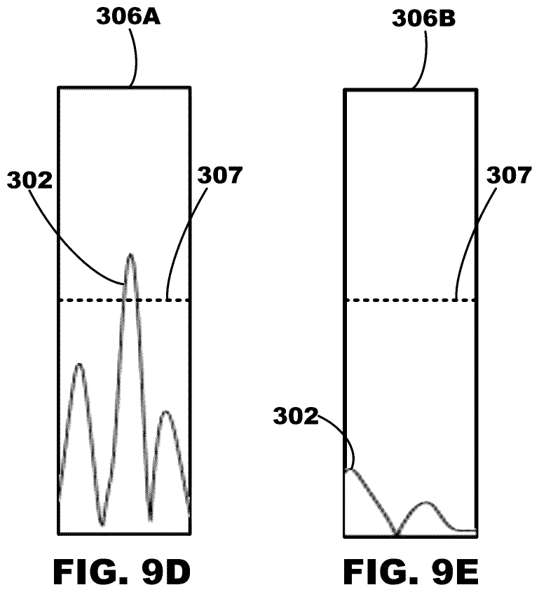

Thus, a single channel of cardiac electrical activity may be provided or obtained 202. The single channel of cardiac electrical activity may be described as a single signal (e.g., sensed or measured voltage) over time. An illustrative single channel of cardiac electrical activity 300 that was measured in a pre-clinical animal study using a right atrial electrode such as, for example, the distal housing-based electrode 22 and the non-tissue piercing electrode(s) 322, and a right atrial blood pool electrode such as, for example, the proximal housing-based electrodes 24, 324 is depicted in FIG. 9A.

Before utilizing any further processes to determine atrial and ventricular events within the single channel of electrical activity 300, the single channel may be pre-processed 204 using one or more signal processing techniques, algorithms, or procedures such as, for example, filtering, amplification, rectification, etc. For example, as shown in the FIG. 9B, the single channel of electrical activity 300 has been filtered, amplified, and rectified resulting in a single channel of processed electrical activity 302. Additionally, the illustrative methods and processes described herein may utilize various comparisons to thresholds, which may be described in terms of being greater than, greater than or equal to, less than, and less than or equal to such thresholds. It is to be understood that such comparisons may utilize the rectified electrical activity, and thus, may utilize the absolute value of the electrical activity. In other embodiments, the monitored electrical activity may not be rectified, and in such embodiments, the threshold comparisons may the opposite or inverse to what is described herein.

In one or more embodiments, a derivative of the single channel of electrical activity 300, either prior to or after pre-processing 204, may be optionally generated 206 resulting in a single channel of derived electrical activity, which may be further used in the illustrative methods and processes described herein to identify, or determine, atrial and ventricular events. For example, the slew rate, or slope, of the single channel of electrical activity 300 may be generated, and then, utilized to identify, or determine, atrial and ventricular events. The first order derivative may enhance signal amplitude separation if the slew rate of the P-waves is lower than the slew rate of the R-waves.

The method 200 may utilize a sense threshold to identify, or determine, windows within the single channel of obtained and processed electrical activity 302 that contain, or include, atrial and ventricular events. It is to be understood that the sense threshold may vary depending on the selected electrodes used to provide the single channel of electrical activity. In one or more embodiments, the sense threshold may be between about 0.15 millivolts (mV) and about 1.0 mV. In one illustrative embodiment, the sense threshold may be 0.75 mV, such as represented by a dashed line 305 in FIG. 9B. In one or more illustrative embodiments, the sense threshold may be a selected percentage of a maximum amplitude of the sensed electrical activity.

When the electrical activity 302 (e.g., the amplitude thereof) exceeds (e.g., greater than) the sense threshold 208 such illustrated as 305 in FIG. 9B, an event window start time may be determined. A window, or portion, of the electrical activity 302 may then be selected, or identified, 210 starting from the event window start time and ending upon the expiration of an event window time period. The event window time period may be between about 30 milliseconds (ms) and 100 ms. In one embodiment, the event window time period is 40 ms. In one or more embodiment, the event window time period may be greater than or equal to 30 ms, greater than or equal to 40 ms, greater than or equal to 50 ms, greater than or equal to 60 ms, greater than or equal to 70 ms, etc. and/or less than or equal to 100 ms, less than or equal to 90 ms, less than or equal to 80 ms, less than or equal to 65 ms, less than or equal to 55 ms, etc. A plurality of windows, or portions, 306A, 306B, 306C, 306D, 306E, 306F are depicted in FIG. 9C overlaying the single channel of processed electrical activity 302.

The method 200 may then determine whether an atrial event or a ventricular event occurred during the window, or portion of the electrical activity 302 that was selected, or identified, 210. In other words, one or more of the atrial and ventricular events may be determined, or identified, based on the monitored, or obtained, electrical activity 300, 302 over the event time window following the determined, or identified, event window start time. In particular, the method 200 may compare 212 the window, or portion of the electrical activity 302 that was selected, or identified, 210 to an A/V threshold. The A/V threshold may be used to determine whether the electrical activity within the identified window, or portion, is representative of an atrial event or a ventricular event. The A/V threshold may be between about 1.5 mV and about 2.75 mV. In one embodiment, the A/V threshold is 2.25 mV. In one or more embodiments, the AV threshold may be greater than or equal to 1.5 mV, greater than or equal to 1.75 mV, greater than or equal to 2.0 mV, greater than or equal to 2.1 mV, etc. and/or less than or equal to 2.75 mV, less than or equal to 2.5 mV, less than or equal to 2.4 mV, less than or equal to 2.35 mV, etc.

If the maximum amplitude of the single channel of electrical activity within the identified portion, or window, is greater than or equal to the A/V threshold, then the method 200 may determine that the electrical activity within the identified portion, or window, is a ventricular event 214 when the electrical activity was monitored using a ventricular electrode such as, e.g., tip electrodes 42, 342, as a cathode. Further, if the maximum amplitude of the single channel of processed electrical activity within the identified portion, or window, is greater than or equal to the A/V threshold, then the method 200 may determine that the electrical activity within the identified portion, or window, is an atrial event 216 when the electrical activity was monitored using a right atrial electrode such as, e.g., distal housing-based electrode 22, non-tissue piercing electrode(s) 322, and proximal housing-based electrodes 24, 324, as a cathode.

Conversely, if the maximum amplitude of the single channel of processed electrical activity within the identified portion, or window, is less than the A/V threshold, then the method 200 may determine that the electrical activity within the identified portion, or window, is an atrial event 216 when the electrical activity was monitored using a ventricular electrode such as, e.g., tip electrodes 42, 342, as a cathode. Further, if the maximum amplitude of the single channel of processed electrical activity within the identified portion, or window, is less than the A/V threshold, then the method 200 may determine that the electrical activity within the identified portion, or window, is a ventricular event 214 when the electrical activity was monitored using a right atrial electrode such as, e.g., distal housing-based electrode 22, non-tissue piercing electrode(s) 322, and proximal housing-based electrodes 24, 324, as a cathode.

In other words, it is to be understood the comparisons to the A/V threshold 212 may depend upon wherefrom the single channel of cardiac electrical activity is sensed. If the single channel of cardiac electrical activity is sensed proximate (e.g., from tissue of) the right atrium, an atrial event is determined 216 when the maximum amplitude within the window is greater than or equal to the A/V threshold and a ventricular event is determined 214 when the maximum amplitude within the window is less than the A/V threshold. Conversely, if the single channel of cardiac electrical activity is sensed proximate (e.g., from tissue of) the left ventricle, a ventricular event is determined 214 when the maximum amplitude within the window is greater than or equal to the A/V threshold and an atrial event is determined 216 when the maximum amplitude within the window is less than the A/V threshold.

Windows 306A, 306B of the single channel of processed cardiac electrical activity 302 are depicted in FIG. 9D and FIG. 9E, respectively. As shown, an A/V threshold 307 of 2.25 mV is represented by a dashed line. As described herein, the single channel of cardiac electrical activity depicted in in FIGS. 9A-9B was monitored, or measured, using a right atrial electrode and a right atrial blood pool electrode, and thus, was monitored, or measured, proximate (e.g., from the tissue of) the right atrium. As a result, atrial events may be determined when the amplitude (e.g., the maximum or peak amplitude) of the electrical activity within window 306A is greater than or equal to the A/V threshold 307, and ventricular events may be determined when the amplitude (e.g., the maximum or peak amplitude) of the electrical activity within window 306A is less than the A/V threshold 307. As shown, the maximum amplitude of the electrical activity 302 within the window 306A is greater than or equal to the A/V threshold 307 indicating an atrial event occurred within the window 306A, and the maximum amplitude of the electrical activity 302 within the window 306B is less than the A/V threshold 307 indicating a ventricular event occurred within the window 306B.

Thus, the illustrative method 200 may be described as being an amplitude discrimination process where the acquired signal is filtered, amplified, and rectified, and then a sense threshold, that is sensitive enough to sense both P-waves and R-waves, is applied. If the signal crosses the sense threshold, the maximum amplitude of the waveform may be determined within an event time window following the signal crossing. If the amplitude is lower than the A/V threshold, then it may be classified as an atrial event (e.g., P-wave) or a ventricular event (e.g., R-wave) depending on the electrodes used to originally obtain the signal. Further, the A/V threshold may be configured by a user or may be determined automatically based on history of prior sensed cardiac events.

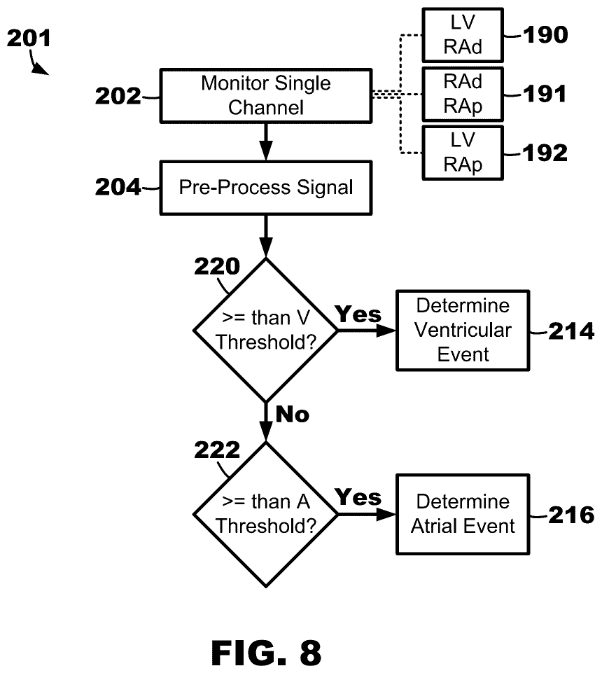

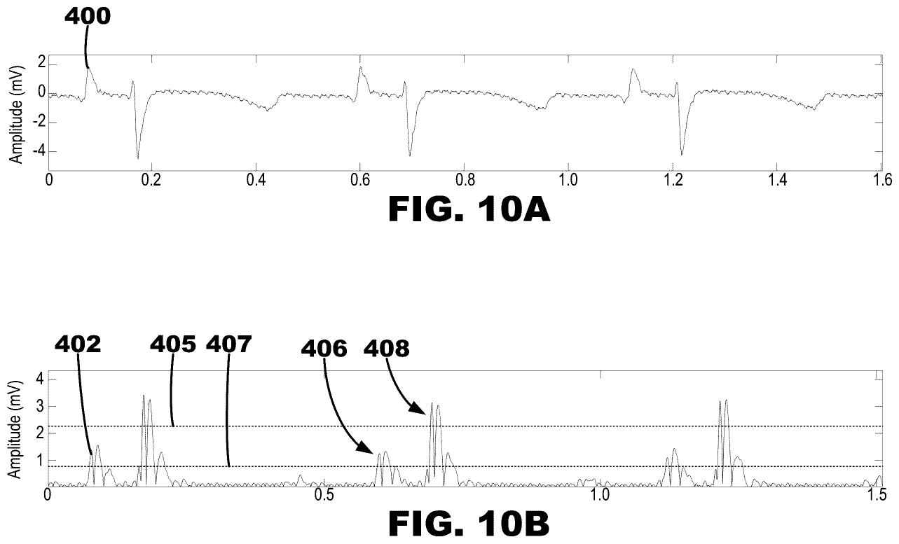

Another illustrative method 201 of atrial and ventricular event identification utilizing a single channel of cardiac electrical activity is depicted in FIG. 8. The method 201 may include some of the same processes as the method 200 depicted in FIG. 7 such as monitoring, or obtaining, a single channel of cardiac electrical activity 202 using an electrode pair 190, 191, 192 and pre-processing the electrical activity 204, and as such, will not be further described herein. In brief, a single channel of cardiac electrical activity may be provided 202 and pre-processed 204. An illustrative single channel of cardiac electrical activity 400 that was measured using a left ventricular electrode such as, for example, the tip electrodes 42, 342, and a right atrial blood pool electrode such as, for example, the proximal housing-based electrodes 24, 324 is depicted in FIG. 10A, and an illustrative single channel of filtered, rectified cardiac electrical activity 402 thereof is depicted in FIG. 10B.

In method 201, a ventricular threshold and an atrial threshold may be utilized to determine, or identify, ventricular and atrial events. It is to be understood that, while the illustrative method 201 is specific to electrical activity, or a signal, monitored using a ventricular electrode such as, e.g., tip electrodes 42, 342, as a cathode, the teachings of method 201 could be applied to electrical activity monitored using an atrial electrode as a cathode by “swapping” the ventricular and atrial thresholds. Additionally, it is to be understood that the ventricular threshold and atrial threshold may vary depending on the selected electrodes used to provide the single channel of electrical activity.

The ventricular threshold may be between about 1.5 mV and about 2.75 mV. In one embodiment, the ventricular threshold is 2.25 mV. In one or more embodiments, the ventricular threshold may be greater than or equal to 1.5 mV, greater than or equal to 1.75 mV, greater than or equal to 2.0 mV, greater than or equal to 2.1 mV, etc. and/or less than or equal to 2.75 mV, less than or equal to 2.5 mV, less than or equal to 2.4 mV, less than or equal to 2.35 mV, etc.

When the electrical activity 402 exceeds or is equal to (e.g., is greater than or equal to) the ventricular threshold 220, then the method 200 may determine that the electrical activity is a ventricular event 214. When the electrical activity 402 is less than the ventricular threshold 220, then the method 200 may determine that the electrical activity is not a ventricular event and may proceed to determining whether the electrical activity is an atrial event.

The atrial threshold may be between about 0.15 mV and about 1.0 mV. In one embodiment, the atrial threshold is 0.75 mV. In one or more embodiments, the atrial threshold may be greater than or equal to 0.5 mV, greater than or equal to 0.6 mV, greater than or equal to 0.7 mV, etc. and/or less than or equal to 1.0 mV, less than or equal to 0.9 mV, less than or equal to 0.8 mV, etc. When the electrical activity 402 (e.g., the amplitude thereof) exceeds or is equal to (e.g., greater than or equal to) the atrial threshold 222, then the method 200 may determine that the electrical activity is an atrial event 216. When the electrical activity 402 is less than the atrial threshold 222, then the method 200 may determine that the electrical activity is not a ventricular nor an atrial event.

A ventricular threshold 405 and an atrial threshold 407, each represented by dashed lines, are depicted in FIG. 10B. As shown, an atrial event 406 is determined when the electrical activity, or signal, 402 is less than the ventricular threshold 405 but greater or equal to the atrial threshold 407, and a ventricular event 408 is determined when the electrical activity, or signal, 402 is greater than the ventricular threshold 405.

Thus, the illustrative method 201 may be described as being a dual sense threshold processes where the acquired signal is filtered, amplified, and rectified, and any signal that crosses an atrial threshold and does not cross a ventricular threshold may be identified as an atrial sense. Further, any signal above the ventricular threshold may be identified, or determined, as a ventricular sense. Each of the atrial threshold and ventricular threshold may be configured by a user or may be computed and automatically adjusted based on history of prior sensed events.

Additionally, the illustrative method 201 may be described as disregarding, or filtering out, undesirable portions of single channel of cardiac electrical activity when attempting to identify atrial and ventricular events. For instance, the atrial threshold and ventricular thresholds may, in effect, be utilized to remove the electrical activity that is unrelated to atrial or ventricular events when analyzing for each. As a result, less processing power may be utilized to determine, or identify, atrial or ventricular events because the unrelated, undesired portions of the single channel of cardiac electrical activity will be not further considered or analyzed.

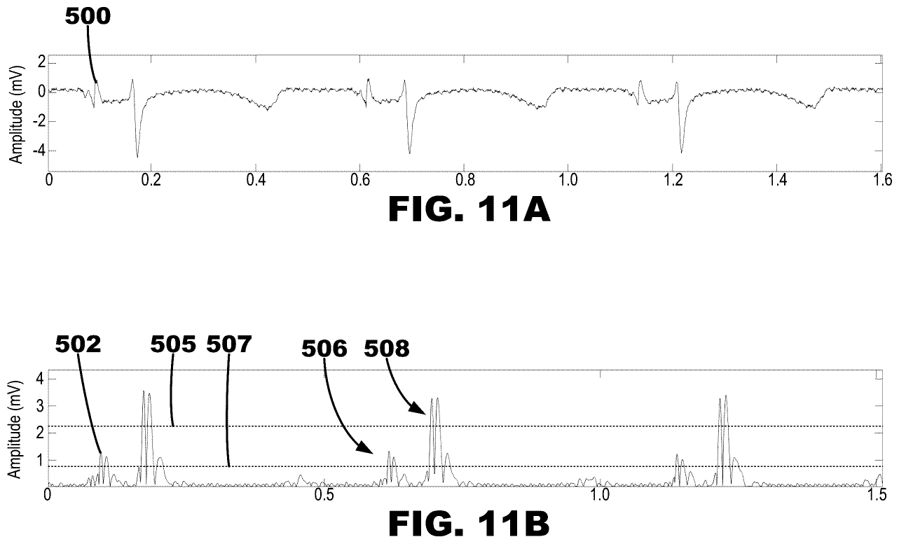

An illustrative single channel of cardiac electrical activity 500 that was measured using a left ventricular electrode such as, for example, the tip electrodes 42, 342, and a right atrial electrode such as, for example, the distal housing-based electrode 22 and non-tissue piercing, electrodes is depicted in FIG. 11A, and an illustrative single channel of filtered, rectified cardiac electrical activity 502 thereof is depicted in FIG. 11B. A ventricular threshold 505 and an atrial threshold 507, each represented by dashed lines, are also depicted in FIG. 11B in accordance with the method 201 depicted in FIG. 8. As shown, an atrial event 506 is determined when the electrical activity, or signal, 502 is less than the ventricular threshold 505 but greater or equal to the atrial threshold 507, and a ventricular event 508 is determined when the electrical activity, or signal, 502 is greater than the ventricular threshold 505.

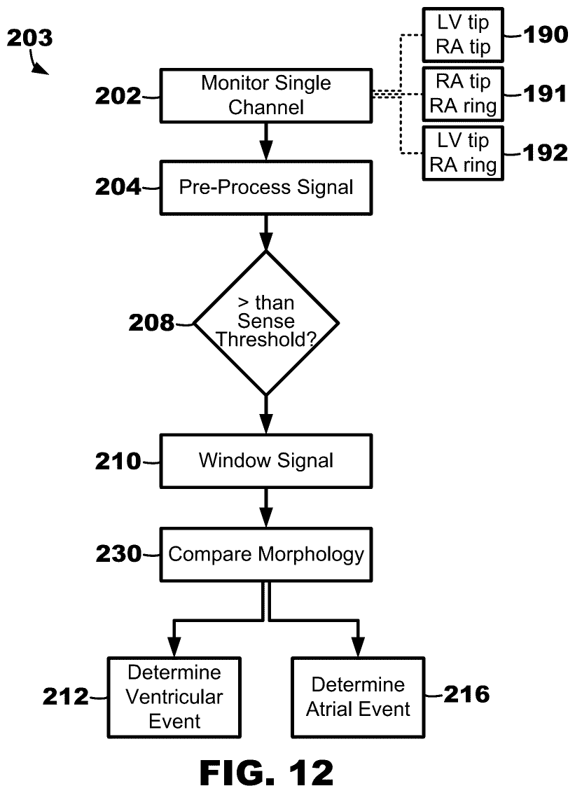

Another illustrative method 203 of atrial and ventricular event identification utilizing a single channel of cardiac electrical activity is depicted in FIG. 12. The method 203 may include some of the same processes as the method 200 depicted in FIG. 7 such as monitoring, or sensing, a single channel of cardiac electrical activity 202 using one or more of electrode pairs 190, 191, 192, pre-processing the electrical activity 204, comparing the electrical activity to the sense threshold 208, and windowing the electrical activity 210, and as such, will not be further described herein. In brief, a single channel of cardiac electrical activity may be provided 202, pre-processed 204, and, utilizing a sense threshold 208, various windows, or portions, of the single channel of electrical activity may be identified, or selected, 210 for further analysis such as determining, or identifying, atrial and ventricular events.

In method 203, the identified window, or portion, of the single channel of electrical activity may be identified, or determined, to be atrial or ventricular events using morphology discrimination or comparison 230. More specifically, the monitored electrical activity within the identified window, or portion, of the single channel of electrical activity may be compared to an atrial electrical morphology template and/or a ventricular electrical morphology template. The atrial electrical morphology template may be a template of a P-wave, and the ventricular electrical morphology template may be a template of a QRS complex. If the identified window of the single channel of electrical activity substantially matches, or is similar to, the atrial electrical morphology template, then the method 203 may determine that the electrical activity is an atrial event 216. Likewise, if the identified window of the single channel of electrical activity substantially matches, or is similar to, the ventricular electrical morphology template, then the method 203 may determine that the electrical activity is a ventricular event 214. In other words, atrial and ventricular events may be determined based on comparisons of the monitored electrical activity to atrial and ventricular electrical morphology templates. Morphology discrimination or comparison may be further described in U.S. Pat. No. 8,983,586 issued on Mar. 17, 2015, which is incorporated herein by reference in its entirety. Additionally, it is to be understood that the illustrative methods and processes described herein may utilize one or more morphological metrics (e.g., slew rate, polarity, width, area, etc.) to determine whether the monitored electrical activity within the identified window, or portion, of the single channel of electrical activity is, or includes, an atrial event or a ventricular event.

In one or more embodiments of methods 200, 201, 203, although a single channel of cardiac electrical activity is utilized, the single channel may be processed into two signals: the first signal being a processed signal that is pre-processed according to the processes described within respect to pre-processing 204 described herein; and the second signal being a raw, unfiltered wideband signal (in other words, the original signal). It is to be understood that the second signal may still be low-pass and high-pass filtered so as to remove baseline variations and high frequency noise and still be essentially a wide-band signal. The processed signal may be utilized by the sensing 208 and windowing 210 processes, which may result in windows, time periods, or start and end time points that may be applied to the raw, unfiltered wideband signal. In other words, the processed signal may be utilized to select, or identify, windows, or portions, of the raw, unfiltered wideband signal for further analysis to determine atrial and ventricular events as described herein with respect to methods 200, 201, 203.

In other words, the single channel may be split out into a sense filtered signal and a wideband signal. The processing of the sense signal may trigger processing on the wideband signal, where amplitude, slew rate, polarity, width, area, template matching, etc. may be used to identify the waveform as either P-wave or R-wave, with fixed or patient-specific thresholds.

The techniques described in this disclosure, including those attributed to the IMD 10, device 50, IMD 310, and/or various constituent components, may be implemented, at least in part, in hardware, software, firmware, or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices, or other devices. The term “module,” “processor,” or “processing circuitry” may generally refer to any of the foregoing logic circuitry, alone or in combination with other logic circuitry, or any other equivalent circuitry.

Such hardware, software, and/or firmware may be implemented within the same device or within separate devices to support the various operations and functions described in this disclosure. In addition, any of the described units, modules, or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed by processing circuitry and/or one or more processors to support one or more aspects of the functionality described in this disclosure.

All references and publications cited herein are expressly incorporated herein by reference in their entirety for all purposes, except to the extent any aspect incorporated directly contradicts this disclosure. All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The recitation of numerical ranges by endpoints includes all numbers subsumed within that range (e.g., 1 to 5 includes 1, 1.5, 2, 2.75, 3, 3.80, 4, and 5) and any range within that range. Herein, the terms “up to” or “no greater than” a number (e.g., up to 50) includes the number (e.g., 50), and the term “no less than” a number (e.g., no less than 5) includes the number (e.g., 5).

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out at least some functionality (for example, a first medical device may be operatively coupled to another medical device to transmit information in the form of data or to receive data therefrom).

Terms related to orientation, such as “top,” “bottom,” “side,” and “end,” are used to describe relative positions of components and are not meant to limit the orientation of the embodiments contemplated. For example, an embodiment described as having a “top” and “bottom” also encompasses embodiments thereof rotated in various directions unless the content clearly dictates otherwise.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

The term “and/or” means one or all the listed elements or a combination of at least two of the listed elements. The phrases “at least one of” “comprises at least one of,” and “one or more of” followed by a list refers to any one of the items in the list and any combination of two or more items in the list.

### ILLUSTRATIVE EMBODIMENTS

Embodiment Em1: An implantable medical device comprising:
a plurality of electrodes comprising:
   a right atrial electrode positionable within the right atrium of a patient's heart, and
   a tissue-piercing electrode implantable through the right atrium from the triangle of Koch region of the right atrium through the right atrial endocardium and central fibrous body and positionable in contact with one or more of the basal region, septal region, and basal-septal region of the left ventricular myocardium of the patient's heart;
a therapy delivery circuit operably coupled to the one or more electrodes to deliver cardiac therapy to the patient's heart;
a sensing circuit operably coupled to the one or more electrodes to sense electrical activity of the patient's heart; and
a controller comprising processing circuitry operably coupled to the therapy delivery circuit and the sensing circuit, the controller configured to:
   monitor a single channel of electrical activity using one or both of the right atrial electrode positionable within the right atrium to sense electrical activity in the right atrium and the tissue-piercing electrode implantable in one or more of the basal region, septal region, and basal-septal region of the left ventricular myocardium of the patient's heart from the triangle of Koch region of the right atrium through the right atrial endocardium and central fibrous body to sense electrical activity in one or more of the basal region, septal region, and basal-septal region of the left ventricular myocardium; and
identify atrial and ventricular events based on the monitored electrical activity.

Embodiment Em2: A method comprising:
obtaining a single channel of electrical activity using one or both of a right atrial electrode positioned within the right atrium to sense electrical activity in the right atrium and a tissue-piercing electrode implanted in one or more of the basal region, septal region, and basal-septal region of the left ventricular myocardium of the patient's heart from the triangle of Koch region of the right atrium through the right atrial endocardium and central fibrous body to sense electrical activity in one or more of the basal region, septal region, and basal-septal region of the left ventricular myocardium; and
identifying atrial and ventricular events based on the obtained electrical activity.

Embodiment Em3: The device as in embodiment Em1 or the method as in embodiment Em2, wherein monitoring or obtaining the single channel of electrical activity using one or both of the right atrial electrode and the tissue-piercing electrode further comprises obtaining the electrical activity between the right atrial electrode and a right atrial blood pool electrode positioned within the right atrial blood pool of the right atrium of the patient's heart.

Embodiment Em4: The device or method as in any one of embodiments Em1-Em3, wherein monitoring or obtaining the single channel of electrical activity using one or both of the right atrial electrode and the tissue-piercing electrode comprises monitoring or obtaining the electrical activity between the right atrial electrode and the tissue-piercing electrode.

Embodiment Em5: The device or method as in any one of embodiments Em1-Em4, wherein identifying atrial and ventricular events based on the monitored or obtained electrical activity comprises one or more of filtering, amplifying, and rectifying the monitored or obtained electrical activity.

Embodiment Em6: The device or method as in any one of embodiments Em1-Em5, wherein identifying atrial and ventricular events based on the monitored or obtained electrical activity comprises:
determining an event window start time when the monitored or obtained electrical activity exceeds a sense threshold; and
determining one or more of the atrial and ventricular events based on the monitored or obtained electrical activity over an event time window following the event window start time.

Embodiment Em7: The device or method as in any one of embodiments Em1-Em6, wherein monitoring or obtaining the single channel of electrical activity using one or both of the right atrial electrode and the tissue-piercing electrode comprises employing the tissue-piercing electrode as a cathode, wherein identifying atrial and ventricular events based on the monitored or obtained electrical activity comprises:
determining the ventricular events if the monitored or obtained electrical activity is greater than or equal to an atrial/ventricular threshold; and
determining the atrial events if the monitored or obtained electrical activity is less than the atrial/ventricular threshold.

Embodiment Em8: The device or method as in any one of embodiments Em1-Em6, wherein monitoring or obtaining the single channel of electrical activity using one or both of the right atrial electrode and the tissue-piercing electrode comprises employing the right atrial electrode as a cathode, wherein identifying atrial and ventricular events based on the monitored or obtained electrical activity comprises:
determining the atrial events if the monitored or obtained electrical activity is greater than or equal to an atrial/ventricular threshold; and
determining the ventricular events if the monitored or obtained electrical activity is less than the atrial/ventricular threshold.

Embodiment Em9: The device or method as in any one of embodiments Em1-Em6, wherein monitoring or obtaining the single channel of electrical activity using one or both of the right atrial electrode and the tissue-piercing electrode comprises employing the tissue-piercing electrode as a cathode, wherein identifying atrial and ventricular events based on the monitored or obtained electrical activity comprises:
determining the ventricular events if the monitored or obtained electrical activity is greater than or equal to a ventricular threshold; and
determining the atrial events if the monitored or obtained electrical activity is greater than or equal an atrial threshold, wherein the ventricular threshold is greater than the atrial threshold.

Embodiment Em10: The device or method as in any one of embodiments Em1-Em6, wherein monitoring or obtaining the single channel of electrical activity using one or both of the right atrial electrode and the tissue-piercing electrode comprises employing the right atrial electrode as a cathode, wherein identifying atrial and ventricular events based on the monitored or obtained electrical activity comprises:
determining the ventricular events if the monitored or obtained electrical activity is greater than or equal to at least a ventricular threshold; and
determining the atrial events if the monitored or obtained electrical activity is greater than or equal at least an atrial threshold, wherein the atrial threshold is greater than the ventricular threshold.

Embodiment Em11: The device or method as in any one of embodiments Em1-Em6, wherein identifying atrial and ventricular events based on the monitored or obtained electrical activity comprises:
comparing the monitored or obtained electrical activity to one or more of an atrial electrical morphology template and a ventricular electrical morphology template; and
determining the atrial and ventricular events based on the comparison of the monitored or obtained electrical activity to one or more of the atrial electrical morphology template and the ventricular electrical morphology template.

Embodiment Em12: The device or method as in any one of embodiments Em1-Em6, wherein identifying atrial and ventricular events based on the monitored or obtained electrical activity comprises:
filtering the monitored or obtained electrical activity to provide only ventricular electrical activity resulting in a ventricular signal;
determining the ventricular events based on the ventricular signal;
filtering the monitored or obtained electrical activity to provide only atrial electrical activity resulting in an atrial signal; and
determining the atrial events based on the atrial signal.

Embodiment Em13: The device or method as in any one of embodiments Em1-Em12, wherein the right atrial electrode and the tissue-piercing electrode are part of a leadless pacemaker.

Embodiment Em14: The device or method as in embodiment Em13, wherein the leadless pacemaker comprises a housing, wherein the right atrial electrode and the tissue-piercing electrode are leadlessly coupled to the housing.

Embodiment Em15: The device or method as in embodiment Em14, wherein the housing is wholly implantable within the right atrium.

Embodiment Em16: An implantable medical device comprising:
a housing wholly implantable in the right atrium of a patient's heart;
a plurality of electrodes leadlessly fixed to the housing, the plurality of electrodes comprising:
   a right atrial electrode positionable within the right atrium, and
   a tissue-piercing electrode implantable through a patient's right atrium so as to extend toward the left ventricular myocardium of the patient's heart; and
a controller comprising processing circuitry operably coupled the plurality of electrodes and within the housing, the controller configured to:
monitor a single channel of electrical activity using one or both of the right atrial electrode positionable within the right atrium and the tissue-piercing electrode implantable through a patient's right atrium so as to extend toward the left ventricular myocardium of the patient's heart; and
identify atrial and ventricular events based on the monitored electrical activity.

Embodiment Em17: A method comprising:
obtaining, with a fully intracardiac leadless device, a single channel of electrical activity using a right atrial electrode of the leadless device positioned within the right atrium of the patient's heart and a tissue-piercing electrode of the leadless device that extends toward the left ventricular myocardium of the patient's heart; and
identifying atrial and ventricular events based on the obtained electrical activity.

Embodiment Em18: The device as in embodiment Em16 or the method as in embodiment Em17, wherein monitoring or obtaining the single channel of electrical activity using one or both of the right atrial electrode and the tissue-piercing electrode further comprises obtaining the electrical activity between the right atrial electrode and a right atrial blood pool electrode positioned within the right atrial blood pool of the right atrium of the patient's heart.

Embodiment Em19: The device or method as in any one of embodiments Em16-Em18, wherein monitoring or obtaining the single channel of electrical activity using one or both of the right atrial electrode and the tissue-piercing electrode comprises monitoring or obtaining the electrical activity between the right atrial electrode and the tissue-piercing electrode.

Embodiment Em20: The device or method as in any one of embodiments Em16-Em19, wherein identifying atrial and ventricular events based on the monitored or obtained electrical activity comprises one or more of filtering, amplifying, and rectifying the monitored or obtained electrical activity.

Embodiment Em21: The device or method as in any one of embodiments Em16-Em20, wherein identifying atrial and ventricular events based on the monitored or obtained electrical activity comprises:
determining an event window start time when the monitored or obtained electrical activity exceeds a sense threshold; and
determining one or more of the atrial and ventricular events based on the monitored or obtained electrical activity over an event time window following the event window start time.

Embodiment Em22: The device or method as in any one of embodiments Em16-Em21, wherein monitoring or obtaining the single channel of electrical activity using one or both of the right atrial electrode and the tissue-piercing electrode comprises employing the tissue-piercing electrode as a cathode, wherein identifying atrial and ventricular events based on the monitored or obtained electrical activity comprises:
determining the ventricular events if the monitored or obtained electrical activity is greater than or equal to an atrial/ventricular threshold; and
determining the atrial events if the monitored or obtained electrical activity is less than the atrial/ventricular threshold.

Embodiment Em23: The device or method as in any one of embodiments Em16-Em21, wherein identifying atrial and ventricular events based on the monitored or obtained electrical activity comprises:
determining the ventricular events based on a comparison of the monitored or obtained electrical activity to at least a ventricular threshold; and
determining the atrial events based on a comparison of the monitored or obtained electrical activity to at least an atrial threshold.

Embodiment Em24: The device or method as in any one of embodiments Em16-Em21, wherein identifying atrial and ventricular events based on the monitored or obtained electrical activity comprises:
comparing the monitored or obtained electrical activity to one or more of an atrial electrical morphology template and a ventricular electrical morphology template; and
determining the atrial and ventricular events based on the comparison of the monitored or obtained electrical activity to one or more of the atrial electrical morphology template and the ventricular electrical morphology template.

Embodiment Em25: The device or method as in any one of embodiments Em16-Em21, wherein identifying atrial and ventricular events based on the monitored or obtained electrical activity comprises:
filtering the monitored or obtained electrical activity to provide only ventricular electrical activity resulting in a ventricular signal;
determining the ventricular events based on the ventricular signal;
filtering the monitored or obtained electrical activity to provide only atrial electrical activity resulting in an atrial signal; and
determining the atrial events based on the atrial signal.

Embodiment Em26: The device or method as in any one of embodiments Em16-Em21, wherein monitoring or obtaining a single channel of electrical activity comprises employing the tissue-piercing electrode as a cathode, and identifying atrial and ventricular events comprises determining ventricular events by applying a higher threshold than that which is applicable to determining atrial events.

Embodiment Em27: The device or method as in any one of embodiments Em16-Em21, wherein monitoring or obtaining a single channel of electrical activity comprises employing the right atrial electrode as a cathode, and identifying atrial and ventricular events comprises determining atrial events by applying a higher threshold than that which is applicable to determining ventricular events.

This disclosure has been provided with reference to illustrative embodiments and aspects and is not meant to be construed in a limiting sense. As described previously, one skilled in the art will recognize that other various illustrative applications may use the techniques as described herein to take advantage of the beneficial characteristics of the devices, systems, and methods described herein. Various modifications of the illustrative embodiments and aspects, as well as additional embodiments and aspects of the disclosure, will be apparent upon reference to this description.

What is claimed:

1. An implantable medical device comprising:
a plurality of electrodes comprising:
a right atrial electrode positionable within the right atrium of a patient's heart, and
a tissue-piercing electrode implantable through the right atrium from the triangle of Koch region of the right atrium through the right atrial endocardium and central fibrous body and positionable in contact with one or more of the basal region, septal region, and basal-septal region of the left ventricular myocardium of the patient's heart;
a therapy delivery circuit operably coupled to the one or more electrodes to deliver cardiac therapy to the patient's heart;
a sensing circuit operably coupled to the one or more electrodes to sense electrical activity of the patient's heart; and
a controller comprising processing circuitry operably coupled to the therapy delivery circuit and the sensing circuit, the controller configured to:
monitor a single channel of electrical activity using one or both of the right atrial electrode positionable within the right atrium to sense electrical activity in the right atrium and the tissue-piercing electrode implantable in one or more of the basal region, septal region, and basal-septal region of the left ventricular myocardium of the patient's heart from the triangle of Koch region of the right atrium through the right atrial endocardium and central fibrous body to sense electrical activity in one or more of the basal region, septal region, and basal-septal region of the left ventricular myocardium; and
identify atrial and ventricular events based on the monitored electrical activity.

2. The device of claim 1, wherein the plurality of electrodes further comprises a right atrial blood pool electrode positionable within the right atrial blood pool of the right atrium of the patient's heart, wherein monitoring the single channel of electrical activity using one or both of the right atrial electrode and the tissue-piercing electrode comprises monitoring the electrical activity between the right atrial electrode and the right atrial blood pool electrode.

3. The device of claim 1, wherein monitoring the single channel of electrical activity using one or both of the right atrial electrode and the tissue-piercing electrode comprises monitoring the electrical activity between the right atrial electrode and the tissue-piercing electrode.

4. The device of claim 1, wherein identifying atrial and ventricular events based on the monitored electrical activity comprises one or more of filtering, amplifying, and rectifying the monitored electrical activity.

5. The device of claim 1, wherein identifying atrial and ventricular events based on the monitored electrical activity comprises:
determining an event window start time when the monitored electrical activity exceeds a sense threshold; and
determining one or more of the atrial and ventricular events based on the monitored electrical activity over an event time window following the event window start time.

6. The device of claim 1, wherein monitoring the single channel of electrical activity using one or both of the right atrial electrode and the tissue-piercing electrode comprises employing the tissue-piercing electrode as a cathode, wherein identifying atrial and ventricular events based on the monitored electrical activity comprises:
determining the ventricular events if the monitored electrical activity is greater than or equal to an atrial/ventricular threshold; and
determining the atrial events if the monitored electrical activity is less than the atrial/ventricular threshold.

7. The device of claim 1, wherein monitoring the single channel of electrical activity using one or both of the right atrial electrode and the tissue-piercing electrode comprises employing the right atrial electrode as a cathode, wherein identifying atrial and ventricular events based on the monitored electrical activity comprises:
determining the atrial events if the monitored electrical activity is greater than or equal to an atrial/ventricular threshold; and
determining the ventricular events if the monitored electrical activity is less than the atrial/ventricular threshold.

8. The device of claim 1, wherein monitoring the single channel of electrical activity using one or both of the right atrial electrode and the tissue-piercing electrode comprises employing the tissue-piercing electrode as a cathode, wherein identifying atrial and ventricular events based on the monitored electrical activity comprises:
determining the ventricular events if the monitored electrical activity is greater than or equal to a ventricular threshold; and
determining the atrial events if the monitored electrical activity is greater than or equal an atrial threshold, wherein the ventricular threshold is greater than the atrial threshold.

9. The device of claim 1, wherein monitoring the single channel of electrical activity using one or both of the right atrial electrode and the tissue-piercing electrode comprises employing the right atrial electrode as a cathode, wherein identifying atrial and ventricular events based on the monitored electrical activity comprises:
determining the ventricular events if the monitored electrical activity is greater than or equal to at least a ventricular threshold; and
determining the atrial events if the monitored electrical activity is greater than or equal at least an atrial threshold, wherein the atrial threshold is greater than the ventricular threshold.

10. The device of claim 1, wherein identifying atrial and ventricular events based on the monitored electrical activity comprises:
comparing the monitored electrical activity to one or more of an atrial electrical morphology template and a ventricular electrical morphology template; and
determining the atrial and ventricular events based on the comparison of the monitored electrical activity to one or more of the atrial electrical morphology template and the ventricular electrical morphology template.

11. The device of claim 1, wherein identifying atrial and ventricular events based on the monitored electrical activity comprises:
filtering the monitored electrical activity to provide only ventricular electrical activity resulting in a ventricular signal;
determining the ventricular events based on the ventricular signal;
filtering the monitored electrical activity to provide only atrial electrical activity resulting in an atrial signal; and
determining the atrial events based on the atrial signal.

12. The device of claim 1, wherein the device is a leadless pacemaker.

13. The device of claim 1, wherein the device further comprises a housing, wherein the right atrial electrode and the tissue-piercing electrode are leadlessly coupled to the housing.

14. The device of claim 12, wherein the housing is wholly implantable within the right atrium.

15. A method comprising:
obtaining a single channel of electrical activity using one or both of a right atrial electrode positioned within the right atrium to sense electrical activity in the right atrium and a tissue-piercing electrode implanted in one or more of the basal region, septal region, and basal-septal region of the left ventricular myocardium of the patient's heart from the triangle of Koch region of the right atrium through the right atrial endocardium and central fibrous body to sense electrical activity in one or more of the basal region, septal region, and basal-septal region of the left ventricular myocardium; and
identifying atrial and ventricular events based on the obtained electrical activity.

16. The method of claim 15, wherein obtaining the single channel of electrical activity using one or both of the right atrial electrode and the tissue-piercing electrode further comprises obtaining the electrical activity between the right atrial electrode and a right atrial blood pool electrode positioned within the right atrial blood pool of the right atrium of the patient's heart.

17. The method of claim 15, wherein obtaining the single channel of electrical activity using one or both of the right atrial electrode and the tissue-piercing electrode comprises obtaining the electrical activity between the right atrial electrode and the tissue-piercing electrode.

18. The method of claim 15, wherein identifying atrial and ventricular events based on the obtained electrical activity comprises one or more of filtering, amplifying, and rectifying the obtained electrical activity.

19. The method of claim 15, wherein identifying atrial and ventricular events based on the obtained electrical activity comprises:
determining an event window start time when the obtained electrical activity exceeds a sense threshold; and
determining one or more of the atrial and ventricular events based on the obtained electrical activity over an event time window following the event window start time.

20. The method of claim 15, wherein obtaining the single channel of electrical activity using one or both of the right atrial electrode and the tissue-piercing electrode comprises employing the tissue-piercing electrode as a cathode, wherein identifying atrial and ventricular events based on the obtained electrical activity comprises:
determining the ventricular events if the obtained electrical activity is greater than or equal to an atrial/ventricular threshold; and
determining the atrial events if the obtained electrical activity is less than the atrial/ventricular threshold.

21. The method of claim 15, wherein obtaining the single channel of electrical activity using one or both of the right atrial electrode and the tissue-piercing electrode comprises employing the right atrial electrode as a cathode, wherein identifying atrial and ventricular events based on the obtained electrical activity comprises:
determining the atrial events if the obtained electrical activity is greater than or equal to an atrial/ventricular threshold; and
determining the ventricular events if the obtained electrical activity is less than the atrial/ventricular threshold.

22. The method of claim 15, wherein obtaining the single channel of electrical activity using one or both of the right atrial electrode and the tissue-piercing electrode comprises employing the tissue-piercing electrode as a cathode, wherein identifying atrial and ventricular events based on the obtained electrical activity comprises:
determining the ventricular events if the obtained electrical activity is greater than or equal to a ventricular threshold; and
determining the atrial events if the obtained electrical activity is greater than or equal to an atrial threshold, wherein the ventricular threshold is greater than the atrial threshold.

23. The method of claim 15, wherein obtaining the single channel of electrical activity using one or both of the right atrial electrode and the tissue-piercing electrode comprises employing the right atrial electrode as a cathode, wherein identifying atrial and ventricular events based on the obtained electrical activity comprises:
determining the ventricular events if the obtained electrical activity is greater than or equal to a ventricular threshold; and
determining the atrial events if the obtained electrical activity is greater than or equal to an atrial threshold, wherein the atrial threshold is greater than the ventricular threshold.

24. The method of claim 15, wherein identifying atrial and ventricular events based on the obtained electrical activity comprises:
comparing the obtained electrical activity to one or more of an atrial electrical morphology template and a ventricular electrical morphology template; and
determining the atrial and ventricular events based on the comparison of the obtained electrical activity one or more of the atrial electrical morphology template and the ventricular electrical morphology template.

25. The method of claim 15, wherein identifying atrial and ventricular events based on the obtained electrical activity comprises:
filtering the obtained electrical activity to provide only ventricular electrical activity resulting in a ventricular signal;
determining the ventricular events based on the ventricular signal;
filtering the obtained electrical activity to provide only atrial electrical activity resulting in an atrial signal; and
determining the atrial events based on the atrial signal.

26. The method of claim 15, wherein the right atrial electrode and the tissue-piercing electrode are part of a leadless pacemaker.

27. The method of claim 26, wherein the leadless pacemaker comprises a housing, wherein the right atrial electrode and the tissue-piercing electrode are leadlessly coupled to the housing.

28. The method of claim 27, wherein the housing is wholly implantable within the right atrium.

29. A method comprising:
obtaining, with a fully intracardiac leadless device, a single channel of electrical activity using a right atrial electrode of the leadless device positioned within the right atrium of the patient's heart and a tissue-piercing electrode of the leadless device that extends toward the left ventricular myocardium of the patient's heart; and
identifying atrial and ventricular events based on the obtained electrical activity.

30. The method of claim 29, wherein obtaining the single channel of electrical activity using one or both of the right atrial electrode and the tissue-piercing electrode further comprises obtaining the electrical activity between the right atrial electrode and a right atrial blood pool electrode positioned within the right atrial blood pool of the right atrium of the patient's heart.

31. The method of claim 29, wherein obtaining the single channel of electrical activity using one or both of the right atrial electrode and the tissue-piercing electrode comprises obtaining the electrical activity between the right atrial electrode and the tissue-piercing electrode.

32. The method of claim 29, wherein identifying atrial and ventricular events based on the obtained electrical activity comprises one or more of filtering, amplifying, and rectifying the obtained electrical activity.

33. The method of claim 29, wherein identifying atrial and ventricular events based on the obtained electrical activity comprises:
determining an event window start time when the obtained electrical activity exceeds a sense threshold; and
determining one or more of the atrial and ventricular events based on the obtained electrical activity over an event time window following the event window start time.

34. The method of claim 29, wherein obtaining the single channel of electrical activity using one or both of the right atrial electrode and the tissue-piercing electrode comprises employing the tissue-piercing electrode as a cathode, wherein identifying atrial and ventricular events based on the obtained electrical activity comprises:
determining the ventricular events if the obtained electrical activity is greater than or equal to an atrial/ventricular threshold; and
determining the atrial events if the obtained electrical activity is less than the atrial/ventricular threshold.

35. The method of claim 29, wherein identifying atrial and ventricular events based on the obtained electrical activity comprises:
determining the ventricular events based on a comparison of the obtained electrical activity to at least a ventricular threshold; and
determining the atrial events based on a comparison of the obtained electrical activity to at least an atrial threshold.

36. The method of claim 29, wherein identifying atrial and ventricular events based on the obtained electrical activity comprises:
comparing the obtained electrical activity to one or more of an atrial electrical morphology template and a ventricular electrical morphology template; and
determining the atrial and ventricular events based on the comparison of the obtained electrical activity one or more of the atrial electrical morphology template and the ventricular electrical morphology template.

37. The method of claim 29, wherein identifying atrial and ventricular events based on the obtained electrical activity comprises:
filtering the obtained electrical activity to provide only ventricular electrical activity resulting in a ventricular signal;
determining the ventricular events based on the ventricular signal;
filtering the obtained electrical activity to provide only atrial electrical activity resulting in an atrial signal; and
determining the atrial events based on the atrial signal.

38. The method of claim 29, wherein obtaining a single channel of electrical activity comprises employing the tissue-piercing electrode as a cathode, and identifying atrial and ventricular events comprises determining ventricular events by applying a higher threshold than that which is applicable to determining atrial events.

39. The method of claim 29, wherein obtaining a single channel of electrical activity comprises employing the right atrial electrode as a cathode, and identifying atrial and ventricular events comprises determining atrial events by applying a higher threshold than that which is applicable to determining ventricular events.

40. An implantable medical device comprising:
a housing wholly implantable in the right atrium of a patient's heart;
a plurality of electrodes leadlessly fixed to the housing, the plurality of electrodes comprising:
a right atrial electrode positionable within the right atrium, and
a tissue-piercing electrode implantable through a patient's right atrium so as to extend toward the left ventricular myocardium of the patient's heart; and
a controller comprising processing circuitry operably coupled the plurality of electrodes and within the housing, the controller configured to:
monitor a single channel of electrical activity using one or both of the right atrial electrode positionable within the right atrium and the tissue-piercing electrode implantable through a patient's right atrium so as to extend toward the left ventricular myocardium of the patient's heart; and
identify atrial and ventricular events based on the monitored electrical activity.

41. The device of claim 40, wherein the plurality of electrodes further comprises a right atrial blood pool electrode positionable within the right atrial blood pool of the right atrium of the patient's heart, wherein monitoring the single channel of electrical activity using one or both of the right atrial electrode and the tissue-piercing electrode comprises monitoring the electrical activity between the right atrial electrode and the right atrial blood pool electrode.

42. The device of claim 40, wherein monitoring the single channel of electrical activity using one or both of the right atrial electrode and the tissue-piercing electrode comprises monitoring the electrical activity between the right atrial electrode and the tissue-piercing electrode.

43. The device of claim 40, wherein identifying atrial and ventricular events based on the monitored electrical activity comprises one or more of filtering, amplifying, and rectifying the monitored electrical activity.

44. The device of claim 40, wherein identifying atrial and ventricular events based on the monitored electrical activity comprises:
determining an event window start time when the monitored electrical activity exceeds a sense threshold; and
determining one or more of the atrial and ventricular events based on the monitored electrical activity over an event time window following the event window start time.

45. The device of claim 40, wherein monitoring the single channel of electrical activity using one or both of the right atrial electrode and the tissue-piercing electrode comprises employing the tissue-piercing electrode as a cathode, wherein identifying atrial and ventricular events based on the monitored electrical activity comprises:
determining the ventricular events if the monitored electrical activity is greater than or equal to an atrial/ventricular threshold; and
determining the atrial events if the monitored electrical activity is less than the atrial/ventricular threshold.

46. The device of claim 40, wherein identifying atrial and ventricular events based on the monitored electrical activity comprises:
determining the ventricular events based on a comparison of the monitored electrical activity to at least a ventricular threshold; and
determining the atrial events based on a comparison of the monitored electrical activity to at least an atrial threshold.

47. The device of claim 40, wherein identifying atrial and ventricular events based on the monitored electrical activity comprises:
comparing the monitored electrical activity to one or more of an atrial electrical morphology template and a ventricular electrical morphology template; and
determining the atrial and ventricular events based on the comparison of the monitored electrical activity to one or more of the atrial electrical morphology template and the ventricular electrical morphology template.

48. The device of claim 40, wherein identifying atrial and ventricular events based on the monitored electrical activity comprises:
filtering the monitored electrical activity to provide only ventricular electrical activity resulting in a ventricular signal;
determining the ventricular events based on the ventricular signal;
filtering the monitored electrical activity to provide only atrial electrical activity resulting in an atrial signal; and
determining the atrial events based on the atrial signal.

49. The device of claim 40, wherein monitoring a single channel of electrical activity comprises employing the tissue-piercing electrode as a cathode, and identifying atrial and ventricular events comprises determining ventricular events by applying a higher threshold than that which is applicable to determining atrial events.

50. The device of claim 40, wherein monitoring a single channel of electrical activity comprises employing the right atrial electrode as a cathode, and identifying atrial and ventricular events comprises determining atrial events by applying a higher threshold than that which is applicable to determining ventricular events.
